SEQUENCE LISTING (148 sequences)

SEQ ID NO: 1 - DNA, 15 bp, Artificial Sequence
  Synthetic Sequence
gctagacgttagcgt

SEQ ID NO: 2 - DNA, 15 bp, Artificial Sequence
  Synthetic Sequence
gctagatgttagcgt

SEQ ID NO: 3 - DNA, 15 bp, Artificial Sequence
  Synthetic Sequence
gctagacgttagcgt

SEQ ID NO: 4 - DNA, 15 bp, Artificial Sequence
  Synthetic Sequence
gctagacgttagcgt

SEQ ID NO: 5 - DNA, 15 bp, Artificial Sequence
  Synthetic Sequence
gcatgacgttgagct

SEQ ID NO: 6 - DNA, 20 bp, Artificial Sequence
  Synthetic Sequence
atggaaggtccagcgttctc

SEQ ID NO: 7 - DNA, 20 bp, Artificial Sequence
  Synthetic Sequence
atcgactctcgagcgttctc

SEQ ID NO: 8 - DNA, 20 bp, Artificial Sequence
  Synthetic Sequence
atcgactctcgagcgttctc

SEQ ID NO: 9 - DNA, 20 bp, Artificial Sequence
  Synthetic Sequence
atcgactctcgagcgttctc

SEQ ID NO: 10 - DNA, 20 bp, Artificial Sequence
  Synthetic Sequence
atggaaggtccaacgttctc

SEQ ID NO: 11 - DNA, 20 bp, Artificial Sequence
  Synthetic Sequence
gagaacgctggaccttccat

SEQ ID NO: 12 - DNA, 20 bp, Artificial Sequence
  Synthetic Sequence
gagaacgctcgaccttccat

SEQ ID NO: 13 - DNA, 20 bp, Artificial Sequence
  Synthetic Sequence
gagaacgctcgaccttcgat

SEQ ID NO: 14 - DNA, 20 bp, Artificial Sequence
  Synthetic Sequence
gagaacgctggaccttccat

SEQ ID NO: 15 - DNA, 20 bp, Artificial Sequence
  Synthetic Sequence
gagaacgatggaccttccat

SEQ ID NO: 16 - DNA, 20 bp, Artificial Sequence
  Synthetic Sequence
gagaacgctccagcactgat

SEQ ID NO: 17 - DNA, 20 bp, Artificial Sequence
  Synthetic Sequence
tccatgtcggtcctgatgct

SEQ ID NO: 18 - DNA, 20 bp, Artificial Sequence
  Synthetic Sequence
tccatgtcggtcctgatgct

SEQ ID NO: 19 - DNA, 20 bp, Artificial Sequence
  Synthetic Sequence
tccatgacgttcctgatgct

SEQ ID NO: 20 - DNA, 20 bp, Artificial Sequence
  Synthetic Sequence
tccatgtcggtcctgctgat

SEQ ID NO: 21 - DNA, 8 bp, Artificial Sequence
  Synthetic Sequence
tcaacgtt

SEQ ID NO: 22 - DNA, 8 bp, Artificial Sequence
  Synthetic Sequence
tcagcgct

SEQ ID NO: 23 - DNA, 8 bp, Artificial Sequence
  Synthetic Sequence
tcatcgat

SEQ ID NO: 24 - DNA, 8 bp, Artificial Sequence
  Synthetic Sequence
tcttcgaa

SEQ ID NO: 25 - DNA, 7 bp, Artificial Sequence
  Synthetic Sequence
caacgtt

SEQ ID NO: 26 - DNA, 8 bp, Artificial Sequence
  Synthetic Sequence
ccaacgtt

SEQ ID NO: 27 - DNA, 8 bp, Artificial Sequence
  Synthetic Sequence
aacgttct

SEQ ID NO: 28 - DNA, 8 bp, Artificial Sequence
  Synthetic Sequence
tcaacgtc

SEQ ID NO: 29 - DNA, 20 bp, Artificial Sequence
  Synthetic Sequence
atggactctccagcgttctc

SEQ ID NO: 30 - DNA, 20 bp, Artificial Sequence
  Synthetic Sequence
atggaaggtccaacgttctc

SEQ ID NO: 31 - DNA, 20 bp, Artificial Sequence
  Synthetic Sequence
atcgactctcgagcgttctc

SEQ ID NO: 32 - DNA, 20 bp, Artificial Sequence
  Synthetic Sequence
atggaggctccatcgttctc

SEQ ID NO: 33 - DNA, 20 bp, Artificial Sequence
  Synthetic Sequence
atcgactctcgagcgttctc

SEQ ID NO: 34 - DNA, 20 bp, Artificial Sequence
  Synthetic Sequence
atcgactctcgagcgttctc

SEQ ID NO: 35 - DNA, 20 bp, Artificial Sequence
  Synthetic Sequence
tccatgtcggtcctgatgct

SEQ ID NO: 36 - DNA, 20 bp, Artificial Sequence
  Synthetic Sequence
tccatgccggtcctgatgct

SEQ ID NO: 37 - DNA, 20 bp, Artificial Sequence
  Synthetic Sequence
tccatggcggtcctgatgct

SEQ ID NO: 38 - DNA, 20 bp, Artificial Sequence
  Synthetic Sequence
tccatgacggtcctgatgct

SEQ ID NO: 39 - DNA, 20 bp, Artificial Sequence
  Synthetic Sequence
tccatgtcgatcctgatgct

SEQ ID NO: 40 - DNA, 20 bp, Artificial Sequence
  Synthetic Sequence
tccatgtcgctcctgatgct

SEQ ID NO: 41 - DNA, 20 bp, Artificial Sequence
  Synthetic Sequence
tccatgtcgtccctgatgct

SEQ ID NO: 42 - DNA, 20 bp, Artificial Sequence
  Synthetic Sequence
tccatgacgtgcctgatgct

SEQ ID NO: 43 - DNA, 20 bp, Artificial Sequence
  Synthetic Sequence
tccataacgttcctgatgct

SEQ ID NO: 44 - DNA, 20 bp, Artificial Sequence
  Synthetic Sequence
tccatgacgtccctgatgct

SEQ ID NO: 45 - DNA, 20 bp, Artificial Sequence
  Synthetic Sequence
tccatcacgtgcctgatgct

SEQ ID NO: 46 - DNA, 19 bp, Artificial Sequence
  Synthetic Sequence
ggggtcaacgttgacgggg

SEQ ID NO: 47 - DNA, 19 bp, Artificial Sequence
  Synthetic Sequence
ggggtcagtcgtgacgggg

SEQ ID NO: 48 - DNA, 15 bp, Artificial Sequence
  Synthetic Sequence
gctagacgttagtgt

SEQ ID NO: 49 - DNA, 20 bp, Artificial Sequence
  Synthetic Sequence
tccatgtcgttcctgatgct

SEQ ID NO: 50 - DNA, 24 bp, Artificial Sequence
  Synthetic Sequence
accatggacgatctgtttcccctc

SEQ ID NO: 51 - DNA, 18 bp, Artificial Sequence
  Synthetic Sequence
tctcccagcgtgcgccat

SEQ ID NO: 52 - DNA, 24 bp, Artificial Sequence
  Synthetic Sequence
accatggacgaactgtttcccctc

SEQ ID NO: 53 - DNA, 24 bp, Artificial Sequence
  Synthetic Sequence
accatggacgagctgtttcccctc

SEQ ID NO: 54 - DNA, 24 bp, Artificial Sequence
  Synthetic Sequence
accatggacgacctgtttcccctc

SEQ ID NO: 55 - DNA, 24 bp, Artificial Sequence
  Synthetic Sequence
accatggacgtactgtttcccctc

SEQ ID NO: 56 - DNA, 24 bp, Artificial Sequence
  Synthetic Sequence
accatggacggtctgtttcccctc

SEQ ID NO: 57 - DNA, 24 bp, Artificial Sequence
  Synthetic Sequence
accatggacgttctgtttcccctc

SEQ ID NO: 58 - DNA, 15 bp, Artificial Sequence
  Synthetic Sequence
cacgttgaggggcat

SEQ ID NO: 59 - DNA, 12 bp, Artificial Sequence
  Synthetic Sequence
tcagcgtgcgcc

SEQ ID NO: 60 - DNA, 17 bp, Artificial Sequence
  Synthetic Sequence
atgacgttcctgacgtt

SEQ ID NO: 61 - DNA, 17 bp, Artificial Sequence
  Synthetic Sequence
tctcccagcgggcgcat

SEQ ID NO: 62 - DNA, 20 bp, Artificial Sequence
  Synthetic Sequence
tccatgtcgttcctgtcgtt

SEQ ID NO: 63 - DNA, 20 bp, Artificial Sequence
  Synthetic Sequence
tccatagcgttcctagcgtt

SEQ ID NO: 64 - DNA, 21 bp, Artificial Sequence
  Synthetic Sequence
tcgtcgctgtctccccttctt

SEQ ID NO: 65 - DNA, 19 bp, Artificial Sequence
  Synthetic Sequence
tcctgacgttcctgacgtt

SEQ ID NO: 66 - DNA, 19 bp, Artificial Sequence
  Synthetic Sequence
tcctgtcgttcctgtcgtt

SEQ ID NO: 67 - DNA, 20 bp, Artificial Sequence
  Synthetic Sequence
tccatgtcgtttttgtcgtt

SEQ ID NO: 68 - DNA, 20 bp, Artificial Sequence
  Synthetic Sequence
tcctgtcgttccttgtcgtt

SEQ ID NO: 69 - DNA, 20 bp, Artificial Sequence
  Synthetic Sequence
tccttgtcgttcctgtcgtt

SEQ ID NO: 70 - DNA, 20 bp, Artificial Sequence
  Synthetic Sequence
tcctgtcgttttttgtcgtt

SEQ ID NO: 71 - DNA, 21 bp, Artificial Sequence
  Synthetic Sequence
tcgtcgctgtctgcccttctt

SEQ ID NO: 72 - DNA, 21 bp, Artificial Sequence
  Synthetic Sequence
tcgtcgctgttgtcgtttctt

SEQ ID NO: 73 - DNA, 20 bp, Artificial Sequence
  Synthetic Sequence
tccatgcgtgcgtgcgtttt

SEQ ID NO: 74 - DNA, 20 bp, Artificial Sequence
  Synthetic Sequence
tccatgcgttgcgttgcgtt

SEQ ID NO: 75 - DNA, 20 bp, Artificial Sequence
  Synthetic Sequence
tccacgacgttttcgacgtt

SEQ ID NO: 76 - DNA, 20 bp, Artificial Sequence
  Synthetic Sequence
tcgtcgttgtcgttgtcgtt

SEQ ID NO: 77 - DNA, 24 bp, Artificial Sequence
  Synthetic Sequence
tcgtcgttttgtcgttttgtcgtt

SEQ ID NO: 78 - DNA, 22 bp, Artificial Sequence
  Synthetic Sequence
tcgtcgttgtcgttttgtcgtt

SEQ ID NO: 79 - DNA, 21 bp, Artificial Sequence
  Synthetic Sequence
gcgtgcgttgtcgttgtcgtt

SEQ ID NO: 80 - DNA, 21 bp, Artificial Sequence
  Synthetic Sequence
tgtcgtttgtcgtttgtcgtt

SEQ ID NO: 81 - DNA, 25 bp, Artificial Sequence
  Synthetic Sequence
tgtcgttgtcgttgtcgttgtcgtt

SEQ ID NO: 82 - DNA, 19 bp, Artificial Sequence
  Synthetic Sequence
tgtcgttgtcgttgtcgtt

SEQ ID NO: 83 - DNA, 14 bp, Artificial Sequence
  Synthetic Sequence
tcgtcgtcgtcgtt

SEQ ID NO: 84 - DNA, 13 bp, Artificial Sequence
  Synthetic Sequence
tgtcgttgtcgtt

SEQ ID NO: 85 - DNA, 20 bp, Artificial Sequence
  Synthetic Sequence
tccatagcgttcctagcgtt

SEQ ID NO: 86 - DNA, 20 bp, Artificial Sequence
  Synthetic Sequence
tccatgacgttcctgacgtt

SEQ ID NO: 87 - DNA, 6 bp, Artificial Sequence
  Synthetic Sequence
gtcgyt

SEQ ID NO: 88 - DNA, 7 bp, Artificial Sequence
  Synthetic Sequence
tgtcgyt

SEQ ID NO: 89 - DNA, 18 bp, Artificial Sequence
  Synthetic Sequence
agctatgacgttccaagg

SEQ ID NO: 90 - DNA, 20 bp, Artificial Sequence
  Synthetic Sequence
tccatgacgttcctgacgtt

SEQ ID NO: 91 - DNA, 20 bp, Artificial Sequence
  Synthetic Sequence
atcgactctcgaacgttctc

SEQ ID NO: 92 - DNA, 20 bp, Artificial Sequence
  Synthetic Sequence
tccatgtcggtcctgacgca

SEQ ID NO: 93 - DNA, 8 bp, Artificial Sequence
  Synthetic Sequence
tcttcgat

SEQ ID NO: 94 - DNA, 20 bp, Artificial Sequence
  Synthetic Sequence
ataggaggtccaacgttctc

SEQ ID NO: 95 - DNA, 15 bp, Artificial Sequence
  Synthetic Sequence
gctagaggggagggt

SEQ ID NO: 96 - DNA, 15 bp, Artificial Sequence
  Synthetic Sequence
gctagatgttagggg

SEQ ID NO: 97 - DNA, 15 bp, Artificial Sequence
  Synthetic Sequence
gctagaggggagggt

SEQ ID NO: 98 - DNA, 15 bp, Artificial Sequence
  Synthetic Sequence
gctagaggggagggt

SEQ ID NO: 99 - DNA, 15 bp, Artificial Sequence
  Synthetic Sequence
gcatgagggggagct

SEQ ID NO: 100 - DNA, 20 bp, Artificial Sequence
  Synthetic Sequence
atggaaggtccagggggctc

SEQ ID NO: 101 - DNA, 20 bp, Artificial Sequence
  Synthetic Sequence
atggactctggagggggctc

SEQ ID NO: 102 - DNA, 20 bp, Artificial Sequence
  Synthetic Sequence
atggactctggagggggctc

SEQ ID NO: 103 - DNA, 20 bp, Artificial Sequence
  Synthetic Sequence
atggactctggagggggctc

SEQ ID NO: 104 - DNA, 20 bp, Artificial Sequence
  Synthetic Sequence
atggaaggtccaaggggctc

SEQ ID NO: 105 - DNA, 20 bp, Artificial Sequence
  Synthetic Sequence
gagaaggggggaccttccat

SEQ ID NO: 106 - DNA, 20 bp, Artificial Sequence
  Synthetic Sequence
gagaaggggggaccttccat

SEQ ID NO: 107 - DNA, 20 bp, Artificial Sequence
  Synthetic Sequence
gagaaggggggaccttggat

SEQ ID NO: 108 - DNA, 20 bp, Artificial Sequence
  Synthetic Sequence
gagaaggggggaccttccat

SEQ ID NO: 109 - DNA, 20 bp, Artificial Sequence
  Synthetic Sequence
gagaaggggggaccttccat

SEQ ID NO: 110 - DNA, 20 bp, Artificial Sequence
  Synthetic Sequence
gagaaggggccagcactgat

SEQ ID NO: 111 - DNA, 20 bp, Artificial Sequence
  Synthetic Sequence
tccatgtggggcctgatgct

SEQ ID NO: 112 - DNA, 20 bp, Artificial Sequence
  Synthetic Sequence
tccatgtggggcctgatgct

SEQ ID NO: 113 - DNA, 20 bp, Artificial Sequence
  Synthetic Sequence
tccatgaggggcctgatgct

SEQ ID NO: 114 - DNA, 20 bp, Artificial Sequence
  Synthetic Sequence
tccatgtggggcctgctgat

SEQ ID NO: 115 - DNA, 20 bp, Artificial Sequence
  Synthetic Sequence
atggactctccggggttctc

SEQ ID NO: 116 - DNA, 20 bp, Artificial Sequence
  Synthetic Sequence
atggaaggtccggggttctc

SEQ ID NO: 117 - DNA, 20 bp, Artificial Sequence
  Synthetic Sequence
atggactctggaggggtctc

SEQ ID NO: 118 - DNA, 20 bp, Artificial Sequence
  Synthetic Sequence
atggaggctccatggggctc

SEQ ID NO: 119 - DNA, 20 bp, Artificial Sequence
  Synthetic Sequence
atggactctggggggttctc

SEQ ID NO: 120 - DNA, 20 bp, Artificial Sequence
  Synthetic Sequence
atggactctggggggttctc

SEQ ID NO: 121 - DNA, 20 bp, Artificial Sequence
  Synthetic Sequence
tccatgtgggtggggatgct

SEQ ID NO: 122 - DNA, 20 bp, Artificial Sequence
  Synthetic Sequence
tccatgcgggtggggatgct

SEQ ID NO: 123 - DNA, 20 bp, Artificial Sequence
  Synthetic Sequence
tccatgggggtcctgatgct

SEQ ID NO: 124 - DNA, 20 bp, Artificial Sequence
  Synthetic Sequence
tccatgggggtcctgatgct

SEQ ID NO: 125 - DNA, 20 bp, Artificial Sequence
  Synthetic Sequence
tccatgtggggcctgatgct

SEQ ID NO: 126 - DNA, 20 bp, Artificial Sequence
  Synthetic Sequence
tccatgtggggcctgatgct

SEQ ID NO: 127 - DNA, 20 bp, Artificial Sequence
  Synthetic Sequence
tccatggggtccctgatgct

SEQ ID NO: 128 - DNA, 20 bp, Artificial Sequence
  Synthetic Sequence
tccatggggtgcctgatgct

SEQ ID NO: 129 - DNA, 20 bp, Artificial Sequence
  Synthetic Sequence
tccatggggttcctgatgct

SEQ ID NO: 130 - DNA, 20 bp, Artificial Sequence
  Synthetic Sequence
tccatggggtccctgatgct

SEQ ID NO: 131 - DNA, 20 bp, Artificial Sequence
  Synthetic Sequence
tccatcgggggcctgatgct

SEQ ID NO: 132 - DNA, 14 bp, Artificial Sequence
  Synthetic Sequence
gctagagggagtgt

SEQ ID NO: 133 - DNA, 20 bp, Artificial Sequence
  Synthetic Sequence
gggggggggggggggggggg

SEQ ID NO: 134 - DNA, 21 bp, Artificial Sequence
  Synthetic Sequence
actgacagactgacagactga

SEQ ID NO: 135 - DNA, 21 bp, Artificial Sequence
  Synthetic Sequence
agtgacagacagacacactga

SEQ ID NO: 136 - DNA, 21 bp, Artificial Sequence
  Synthetic Sequence
actgacagactgatagaccca

SEQ ID NO: 137 - DNA, 21 bp, Artificial Sequence
  Synthetic Sequence
agtgagagactgcaagactga

SEQ ID NO: 138 - DNA, 21 bp, Artificial Sequence
  Synthetic Sequence
aatgccagtccgacaggctga

SEQ ID NO: 139 - DNA, 21 bp, Artificial Sequence
  Synthetic Sequence
ccagaacagaagcaatggatg

SEQ ID NO: 140 - DNA, 21 bp, Artificial Sequence
  Synthetic Sequence
cctgaacagaagccatggatg

SEQ ID NO: 141 - DNA, 21 bp, Artificial Sequence
  Synthetic Sequence
gcagaacagaagacatggatg

SEQ ID NO: 142 - DNA, 21 bp, Artificial Sequence
  Synthetic Sequence
ccacaacacaagcaatggata

SEQ ID NO: 143 - DNA, 21 bp, Artificial Sequence
  Synthetic Sequence
aagctagccagctagctagca

SEQ ID NO: 144 - DNA, 21 bp, Artificial Sequence
  Synthetic Sequence
cagctagccacctagctagca

SEQ ID NO: 145 - DNA, 21 bp, Artificial Sequence
  Synthetic Sequence
aagctaggcagctaactagca

SEQ ID NO: 146 - DNA, 21 bp, Artificial Sequence
  Synthetic Sequence
gagctagcaagctagctagga

SEQ ID NO: 147 - DNA, 24 bp, Artificial Sequence
  Synthetic
tcgtcgttttgtcgttttgtcgtt

SEQ ID NO: 148 - DNA, 24 bp, Artificial Sequence
  Synthetic
tttttttttttttttttttttttt

(19) United States
(12) Patent Application Publication
(10) Pub. No.: US 2002/0198165 A1
(43) Pub. Date: Dec. 26, 2002

(54) Nucleic acids for the prevention and treatment of gastric ulcers

(76) Inventors: Robert L. Bratzler, Concord, MA (US); Deanna M. Petersen, Newton, MA (US)

Correspondence Address: Maria A. Trevisan, c/o Wolf, Greenfield & Sacks, P.C., Federal Reserve Plaza, 600 Atlantic Avenue, Boston, MA, 02210-2211, US

(21) Appl. No.: 09/920,313

(22) Filed: Aug. 1, 2001

Related U.S. Application Data
(60) Provisional application No. 60/222,248, filed on Aug. 1, 2000.

(51) Int. Cl. A61K 48/00

(52) U.S. Cl. 514/44

(57) ABSTRACT

The invention relates to methods and products for treating gastric ulcers. A nucleic acid and optionally an anti-ulcer agent are administered to a subject to prevent or treat gastric ulcer.

### RELATED APPLICATIONS

This application claims priority under Title 35 §119(e) of the U.S. Provisional Application No. 60/222,248 filed Aug. 1, 2000, and entitled “Nucleic Acids for the Prevention and Treatment of Gastric Ulcers”, the entire contents of which are incorporated herein by reference.

### FIELD OF THE INVENTION

The invention relates to methods, products, and kits for treating and/or preventing gastric ulcers.

### BACKGROUND OF THE INVENTION

Millions of individuals worldwide suffer from ulcers, which are sores or holes in the lining of the stomach or of the duodenum. Common symptoms of gastric ulcer include gnawing or burning pain in the abdomen. The pain can occur at any time but often occurs when the stomach is empty, between meals or in the early morning hours. Other symptoms include nausea, vomiting, loss of appetite, and sometimes bleeding. Pain associated with ulcers is often treated with antacids or by eating food.

### SUMMARY OF THE INVENTION

The invention is based in part on the discovery of a new class of compounds for the treatment and prevention of gastric ulcer. The invention in one aspect is a method for preventing or treating a gastric ulcer by administering to a subject in need thereof an effective amount for preventing or treating a gastric ulcer of a nucleic acid. In other aspects the invention is composition, including a nucleic acid and an anti-ulcer agent, formulated in a pharmaceutically-acceptable carrier and in an effective amount for preventing or treating an ulcer.

According to other aspects the invention is a kit including a nucleic acid, at least one container housing an anti-ulcer agent, and instructions for administering the anti-ulcer agent to a subject having an ulcer or at risk of developing an ulcer.

A nucleic acid is an element of each aspect of the invention. The nucleic acids useful according to the invention are synthetic or natural (isolated) nucleic acids. The nucleic acid may be administered alone or in conjunction with a pharmaceutically-acceptable carrier and optionally other therapeutic agents. In one embodiment, the nucleic acid is an immunostimulatory nucleic acid. The immunostimulatory nucleic acid is any nucleic acid which is capable of modulating an immune response. In some embodiments the immunostimulatory nucleic acid is a CpG nucleic acid having an unmethylated CpG motif, a T-rich nucleic acid, or a poly G nucleic acid. In some embodiments the immunostimulatory nucleic acid is not an H. pylori anti-sense nucleic acid or a vector expressing a gene encoding an H. pylori antigen. In other embodiments the immunostimulatory nucleic acid is an antisense nucleic acid or a vector expressing a gene encoding an H. pylori antigen.

The immunostimulatory nucleic acid may be administered to a subject or formulated in a composition alone or in combination with an anti-ulcer agent. An anti-ulcer agent in some embodiments includes, but is not limited to, an anti-bacterial agent, an oligosaccharide, a somatostatin, a somatostatin agonist, a combination of an H₂ receptor blocker and an acid degradable antibacterial compound, a flavone compound, an imidazopyridazine, a dimethicone, a pyridine compound, a monoglyceride of fatty acids and lauric acid, an N-substituted derivative of 2-(pyridylalkene sulfinyl)benzimidazole, a thymus plant extract, a diphenyl ether phosphate ester, a triclosan, anti-Helicobacter pylori immunoglobulin, a salt of a basic histamine H₂ -receptor antagonist or a solvate thereof, a complex of bismuth with a carboxylic acid, a sulfated glyceroglucolipid, a polypeptide isolated from Streptococcus pneumoniae and Staphylococcus aureus, an antacid, ulcer adherent complex, H₂ receptor blockers/antagonist, proton pump (H⁺, K⁺-ATPase) inhibitor, anti-cholinergic, or an ACE-inhibitor. The anti-ulcer agent in some embodiments is not an anti-bacterial agent.

The anti-bacterial agent may be an antibiotic, such as a broad spectrum antibiotic, a narrow spectrum antibiotic, or a limited spectrum antibiotic. In some embodiments the anti-bacterial agent is a cell wall synthesis inhibitor, cell membrane inhibitor, protein synthesis inhibitor, nucleic acid synthesis or functional inhibitor, competitive inhibitor, amoxicillin; clarithromycin; amoxicillin/clarithromycin combination; metronidazole; tetracycline, or naphthyridine carboxylic acid antibacterial compounds, or combinations thereof.

The antacid in some embodiments includes, but is not limited to, aluminum hydroxide, aluminum carbonate, aluminum phosphate, calcium carbonate, magnesium oxide, magnesium hydroxide, magnesium carbonate, magnesium alginate, magnesium trisilicate, sodium bicarbonate, sodium alginate, magaldrate, simethicone, or combinations thereof.

The ulcer adherent complex in some embodiments includes, but is not limited to, an alpha-D glucopyranoside beta-D fructofuranosyl-octakis-(hydrogen sulfate) aluminum complex such as sucralfate.

The H₂ receptor blockers/antagonist in some embodiments includes, but is not limited to, nizatidine, famotidine, cimetidine, or ranitidine hydrochloride.

The proton pump inhibitor in some embodiments includes, but is not limited to, omeprazole, lansoprazole, or prevpac.

The anti-cholinergic in some embodiments includes, but is not limited to, atropine, belladonna, clidinium, hyoscyamine, pirenzepine, or propantheline.

The ACE-inhibitor in some embodiments includes, but is not limited to, alacepril, alatriopril, altiopril calcium, ancovenin, benazepril, benazepril hydrochloride, benazeprilat, benzazepril, benzoylcaptopril, captopril, captopril-cysteine, captopril-glutathione, ceranapril, ceranopril, ceronapril, cilazapril, cilazaprilat, converstatin, delapril, delapril-diacid, enalapril, enalaprilat, enalkiren, enapril, epicaptopril., foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciurmin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril, zofenoprilat, racemic forms thereof, and pure or substantially pure enantiomers thereof.

The nucleic acid in some embodiments has a nucleotide backbone which includes at least one backbone modification, such as a phosphorothioate modification or other phosphate modification. In some embodiments the modified backbone is a peptide modified oligonucleotide backbone. The nucleotide backbone may be chimeric, or the nucleotide backbone is entirely modified.

The immunostimulatory nucleic acid can have any length greater than 6 nucleotides, but in some embodiments is between 8 and 100 nucleotide residues in length. In other embodiments the nucleic acid comprises at least 20 nucleotides, at least 24 nucleotides, at least 27, nucleotides, or at least 30 nucleotides. The nucleic acid may be single stranded or double stranded. In some embodiments the nucleic acid is isolated and in other embodiments the nucleic acid may be a synthetic nucleic acid.

The CpG nucleic acid in one embodiment contains at least one unmethylated CpG dinucleotide having a sequence including at least the following formula: 5′ X₁ X₂CGX₃ X₄ 3′ wherein C is unmethylated, wherein X₁, X₂, X₃, and X₄ are nucleotides. In one embodiment the 5′ X₁ X₂CGX₃ X₄ 3′ sequence of the CpG nucleic acid is a non-palindromic sequence, and in other embodiments it is a palindromic sequence.

In some embodiments X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, CpT, ApT, TpG, ApG, CpG, TpC, ApC, CpC, TpA, ApA, and CpA. In other embodiments X₁X₂ are GpA or GpT and X₃X₄ are TpT. In yet other embodiments X₁ or X₂ or both are purines and X₃ or X₄ or both are pyrimidines or X₁X₂ are GpA and X₃ or X₄ or both are pyrimidines. In one embodiment X₂ is a T and X₃ is a pyrimidine.

In some embodiments the T rich immunostimulatory nucleic acid is a poly T nucleic acid comprising 5′ TTTT 3′. In yet other embodiments the poly T nucleic acid comprises 5′ X₁ X₂TTTTX₃ X₄ 3′ wherein X₁, X₂, X₃ and X₄ are nucleotides. In some embodiments X₁X₂ is TT and/or X₃X₄ is TT. In other embodiments X₁X₂ is selected from the group consisting of TA, TG, TC, AT, AA, AG, AC, CT, CC, CA, CG, GT, GG, GA, and GC; and/or X₃X₄ is selected from the group consisting of TA, TG, TC, AT, AA, AG, AC, CT, CC, CA, CG, GT, GG, GA, and GC.

The T rich immunostimulatory nucleic acid may have only a single poly T motif or it may have a plurality of poly T nucleic acid motifs. In some embodiments the T rich immunostimulatory nucleic acid comprises at least 2, at least 3, at least 4, at least 5, at least 6, at least 7, or at least 8 T motifs. In other embodiments it comprises at least 2, at least 3, at least 4, at least 5, at least 6, at least 7, or at least 8 CpG motifs. In some embodiments the plurality of CpG motifs and poly T motifs are interspersed.

In yet other embodiments at least one of the plurality of poly T motifs comprises at least 3, at least 4, at least 5, at least 6, at least 7, at least 8, or at least 9 contiguous T nucleotide residues. In other embodiments the plurality of poly T motifs is at least 3 motifs and wherein at least 3 motifs each comprises at least 3 contiguous T nucleotide residues or the plurality of poly T motifs is at least 4 motifs and wherein the at least 4 motifs each comprises at least 3 contiguous T nucleotide residues.

The T rich immunostimulatory nucleic acid may include one or more CpG motifs. The motifs may be methylated or unmethylated. In other embodiments the T rich immunostimulatory nucleic acid is free of one or more CpG dinucleotides.

In other embodiments the T rich immunostimulatory nucleic acid has poly A, poly G, and/or poly C motifs. In other embodiments the T rich immunostimulatory nucleic acid is free of two poly C sequences of at least 3 contiguous C nucleotide residues. Preferably the T rich immunostimulatory nucleic acid is free of two poly A sequences of at least 3 contiguous A nucleotide residues. In other embodiments the T rich immunostimulatory nucleic acid comprises a nucleotide composition of greater than 25% C or greater than 25% A. In yet other embodiments the T rich immunostimulatory nucleic acid is free of poly-C sequences, poly G sequences or poly-A sequences.

In some cases the T rich immunostimulatory nucleic acid may be free of poly T motifs, but rather, comprises a nucleotide composition of greater than 25% T. In other embodiments the T rich immunostimulatory nucleic acid may have poly T motifs and also comprise a nucleotide composition of greater than 25% T. In some embodiments the T rich immunostimulatory nucleic acid comprises a nucleotide composition of greater than 25% T, greater than 30% T, greater than 40% T, greater than 50% T, greater than 60% T, greater than 80% T, or greater than 90% T nucleotide residues.

In some embodiments the poly G nucleic acid comprises: 5′ X₁X₂GGGX₃X₄ 3′ wherein X₁, X₂, X₃, and X₄ are nucleotides. In embodiments at least one of X₃ and X₄ are a G or both of X₃ and X₄ are a G. In other embodiments the poly G nucleic acid comprises the following formula: 5′ GGGNGGG 3′ wherein N represents between 0 and 20 nucleotides. In yet other embodiments the poly G nucleic acid comprises the following formula: 5′ GGGNGGGNGGG 3′ wherein N represents between 0 and 20 nucleotides.

The poly G immunostimulatory nucleic acid may include one or more CpG motifs or T-rich motifs. The CpG motifs may be methylated or unmethylated. In other embodiments the poly G nucleic acid is free of one or more CpG dinucleotides or poly-T motifs.

The nucleic acid and optionally the anti-ulcer agent may be administered by any route known in the art for delivering medicaments. The medicaments may be administered separately or together, in the same pharmaceutical formulation or separate formulations, by the same route or by different routes. In one embodiment the nucleic acid is administered on a routine schedule. In another embodiment the anti-ulcer agent is administered on a routine schedule.

Each of the limitations of the invention can encompass various embodiments of the invention. It is, therefore, anticipated that each of the limitations of the invention involving any one element or combinations of elements can be included in each aspect of the invention.

### DETAILED DESCRIPTION FO THE INVENTION

Many millions of individuals suffer from gastric ulcer worldwide. New methods for preventing the onset or development of gastric ulcer and for treating gastric ulcer once it is developed are described herein. Gastric ulcer, also referred to as peptic ulcer, duodenal ulcer, stomach ulcer, or ulcer, as used herein, refers to a clinical disorder involving a region of inflammation, denudation, ulceration, or other damage in one or more parts of the gastrointestinal tract, including the stomach, small intestine, large intestine or the junctions between each. The actual cause of gastric ulcer is unknown. It has been proposed that gastric ulcer is caused by the production of excess stomach acid and pepsin with a rapid gastric emptying time, which results in mucosal damage from the increased exposure of the duodenum to secreted acids. Another cause of gastric ulcer is believed to be due to increased stomach acid and a breakdown of the complex stomach defenses that normally protect the gastric mucosa from damage by these substances. More recently, the development of gastric ulcers have been linked to infection with Helicobacter pylori (H. pylori). Gastric ulcers may arise as a result of some combination of these components as well as other as yet unknown causes.

H. pylori is a spiral-shaped bacterium which is found in the gastric mucous layer or within the epithelial lining of the stomach. Many more individuals are infected with H. pylori, than actually develop ulcers. About two-thirds of the world's population are believed to be infected with H. pylori, but much fewer experience symptoms associated with gastric ulcer. Individuals infected with H. pylori, however, are believed to have an increased risk of developing gastric abnormalities than uninfected individuals.

Several methods for detecting H. pylori infection in a subject are known and may be used in a clinical setting to determine the presence of H. pylori. Such methods have been described in patents, for instance, U.S. Pat. No. 5,989,840 which describes a diagnostic device for identifying active H. pylori infectious agents in saliva. Other diagnostic tests are commercially available, e.g serological tests that measure specific H. pylori IgG antibodies, breath tests, and biopsy analysis performed during upper esophogastriduodenal endoscopy. Breath tests are accomplished, for instance, by orally administering to a subject a labeled carbon material such as ¹⁴C or ¹³C which is capable of being metabolized by H. pylori and excreted from the subject as CO₂. The labeled C is then detected in the air breathed out by the subject. Biopsy specimens of the stomach and duodenum can be obtained during endoscopy and examined using a biopsy eurease test, histological analysis, and/or biopsy culture of specimens.

The methods described herein are useful for preventing and/or treating gastric ulcer. The terms “prevent” and “preventing” as used herein, refer to inhibiting completely or partially as well as slowing the onset of gastric ulcer. The terms “treat”, “treated”, and “treating” as used herein refer to decreasing the severity of an existing gastric ulcer, as well as, in some cases, completely eliminating the gastric ulcer or inhibiting an increase in the severity of an existing gastric ulcer. Thus, the term “prevention” embraces the use of the compounds of the invention for inhibiting the development of gastric ulcer before it begins or slowing its onset. The term “treatment” embraces the use of the invention for decreasing the severity of the disease or treating a subject in which an ulcer has already formed in order to slow or inhibit altogether the progression of the ulcer.

The nucleic acids are useful in some aspects as a prophylactic for the prevention of a gastric ulcer in a subject at risk of developing an ulcer. A “subject at risk” as used herein is a subject who has any risk of developing an ulcer. For instance, a subject at risk may be a subject who is at risk of being exposed to H. pylori or it may be a subject who has already been infected with H. Pylori but has not yet developed an ulcer. Other persons at risk are those who have had ulcers in the past and thus may develop them again.

In addition to the use of the nucleic acids for prophylactic treatment, the invention also encompasses the use of the nucleic acids for the treatment of a subject having an ulcer. A “subject having an ulcer” is a subject that has actually developed an ulcer as defined above and in some cases but not all may have acute or chronic detectable levels of the H. pylori pathogen in the body.

A “subject” a used herein is a human or non-human vertebrate animal including but not limited to dog, cat, horse, cow, goat, sheep, pig, rabbit, turkey, chicken, primate, rat, and mouse.

In addition to humans, several animals also suffer from ulcers. For instance, gastric ulceration is a serious disease in horses, and is referred to as equine gastric ulcer syndrome (EGUS). This syndrome, which in many cases is symptomatic, may also be asymptomatic and is often associated with focal or multifocal lesions of squamous mucosa, glandular mucosa, or both, and gastritis. Because of the wide range of ulceration sites and degree of severity a scoring system has been developed in order to aid therapy of the horses. The severity ranges from a grade zero ulcer which is associated with inflamed but intact epithelium, to superficial erosions to multiple active hemorrhaging lesions which extend beneath the mucosal surface (a grade three ulcer). If perforation occurs, it is generally fatal to the horse. Gastric ulcers are believed to be an even greater problem in race horses. One postmortem study showed that 66% of all horses examined had gastric ulcers, but that 80% of race horses had gastric ulcers. (Hammond, C. J., et al., Equine Vet. J. 1986, 18:284-287.) Many different causes have been attributed to the development of ulcers in horses, including high levels of acid secretion. In adult horses, microbial agents have not been associated with ulcer development. For instance, helicobacter pylori have not been isolated from horse stomachs and thus are not believed to be a cause of horse ulcers. Instead, the risk factors associated with equine development of ulcers include intensive exercise, diet, physical stress, illness, and medication such as nonsteroidal anti-inflammatory drugs (NSAID). The conventional methods for treating ulcers include the use of drugs such as antacids in order to elevate gastric pH, coating of the ulcer, and supplementing endogenous prostaglandins. The drugs of choice in addition to antacids include histamine H2-receptor antagonists, acid pump inhibitors, sucralfate, synthetic analogs of prostaglandin E₂, such as misophrostol, bismuth, subsalicylate, and prokinetic drugs such as bethanachol.

The FDA Center for Veterinary Medicine has recently approved the first drug specifically for the treatment and prevention of recurrent gastric ulcers in horses and foals greater than four weeks of age. This drug marketed under the name Gastro Guard is a formulation of omeprazole. It is formulated as an oral paste in a calibrated syringe. The immunostimulatory nucleic acids of the invention can be administered alone or in combination with any of these drugs for the treatment and prevention of ulcers in horses. The invention also encompasses, compositions of the immunostimulatory nucleic acids with drugs such as Gastro Guard.

The compounds of the invention may be administered alone or in combination with an anti-ulcer agent. An anti-ulcer agent, as used herein, refers to any compound which is useful for treating gastric ulcer. These compounds include, for instance, any of the compounds listed or described herein as well as any other compounds which have been suggested to be useful for the treatment of ulcer, but specifically exclude antigens of H. pylori. An antigen of H. pylori includes intact H. pylori or fragments of H. pylori which induce a specific immune response against H. pylori. Antigens of H. pylori are described in several references and patents including U.S. Pat. Nos. 6,025,164; 5,814,455; 5,538,729; 5,801,013; and 5,420,014, as well as PCT Published Patent Application numbers WO97/37044 and WO97/19098. Anti-ulcer agents useful according to the invention include, but are not limited to, the following compounds and classes of compounds, an anti-bacterial agent, an antacid, ulcer adherent complex, H₂ receptor blockers/antagonist, proton pump (H⁺, K⁺-ATPase) inhibitor, anti-cholinergic, or an agent for treating H. pylori infection, such as an ACE-inhibitor or other compound. The anti-ulcer agent in some embodiments is not an anti-bacterial agent.

Many types of drugs have been proposed and developed for the treatment of gastric ulcer. Traditionally, these drugs include compounds which block or reduce acid secretion or neutralize the acids. These compounds include antacids, ulcer adherent complex, H₂ receptor blocker/antagonists, proton pump (H⁺, K⁺-ATPase) inhibitors, anti-cholinergics, oligosaccharides, somatostatin or somatostatin agonists, and others. More recently, with the identification of the role of H. pylori infection in developing gastric ulcers, other types of treatments have been proposed. These include, for example, antibiotics, ACE-inhibitors, immunogenic compositions capable of inducing antibodies against H. pylori, specific immunoglobulins derived from animals which have been immunized or exposed to H. pylori, and others. Some commercial compounds which are used for treating gastric ulcer are shown in Table 1 and 2.

TABLE 1
PharmaPipelines: Pipeline Analysis by Therapeutic Category

| | | | | MECHA- |
|---|---|---|---|---|
| | BRAND | GENERIC | INDI- | NISM OF |
| COMPANY | NAME | NAME | CATION | ACTION |
| PHONE | Maalox | Al hydroxide | Acid | Antacid |
| POULENC | | | Disorders | |
| YAMA- | Maalox | Al hydroxide | Acid | Antacid |
| NOUCHI | | | Disorders | |
| KISSEI | Alanta | Aldioxa | Acid | Antacid |
| | | | Disorders | |
| DAIICHI | Muralis | Ecabamide | Acid | Antacid |
| | (DQ2511) | | Disorders | |
| ALTANA | Riopan | Malagdrat | Acid | Antacid |
| | | | Disorders | |
| B. | Gastrozepin | Pirenzepine | Acid | Anti- |
| INGELHEIM | | | Disorders | cholinergic |
| DAIICHI | Neuer | Cetraxate | Acid | Cyto- |
| | | Hhydro- | Disorders | protective |
| | | chloride | | |
| MERCK | Ulcogant | Sucralfate | Acid | Cyto- |
| KGAA | | | Disorders | protective |
| CHUGAN | Ulcerlmin | Sucralfate | Acid | Cyto- |
| | | | Disorders | protective |
| EISAI | Selbex | Teprenone | Acid | Cyto- |
| | | | Disorders | protective |
| TANABE | Cerekinon | Trimebutine | Acid | Cyto- |
| SEIYAKU | | | Disorders | protective |
| TAKEDA | EM-574 | EM-574 | Acid | Digestive |
| | | | Disorders | tract |
| | | | | function |
| | | | | activator |
| SMITHKLINE | Tagamet | Cimetidine | Acid | H2 |
| BEECHAM | | | Disorders | antagonist |
| FUJISAWA | Tagamet | Cimetidine | Acid | H2 |
| | | | Disorders | antagonist |
| B. | Ganor | Famotidine | Acid | H2 |
| INGELHEIM | | | Disorders | antagonist |
| MERCK | Pepcid | Famotidine | Acid | H2 |
| | | | Disorders | antagonist |
| YAMA- | Gaster or | Famotidine | Acid | H2 |
| NOUCHI | Pepcid | | Disorders | antagonist |
| LILLY | Axid | Nizatidine | Acid | H2 |
| | | | Disorders | antagonist |
| GLAXO | Zantac | Ranitidine | Acid | H2 |
| WELL- | | | Disorders | antagonist |
| COMME | | | | |
| SANKYO | Zantac | Ranitidine | Acid | H2 |
| | | | Disorders | antagonist |
| HOECHST | Roxit | Roxatidine | Acid | H2 |
| | | | Disorders | antagonist |
| TAKEDA | Altat | Roxatidine | Acid | H2 |
| | | | Disorders | antagonist |
| JOHNSON & | Propulsid | Cisapride | Acid | Prokinetic |
| JOHNSON | | | Disorders | |
| JOHNSON & | Norcisapride | Norcisapride | Acid | Prokinetic |
| JOHNSON | | (+) | Disorders | |
| SEPRACOR | Norcisapride | Norcisapride | Acid | Prokinetic |
| | | (+) | Disorders | |
| SCHERING | Norcisapride | Norcisapride | Acid | Prokinetic |
| PLOUGH | | (+) | Disorders | |
| ONO | Ronok | Omoprostil | Acid | Prosta- |
| | | | Disorders | glandin |
| EISAI | Pariet | Rabeprazole | Acid | Protease |
| | | | Disorders | inhibitor |
| ABBOTT | Lanzor/ | Lansoprazole | Acid | Proton pump |
| | Prevacid | | Disorders | inhibitor |
| HOECHST | Lansor | Lansoprazole | Acid | Proton pump |
| | | | Disorders | inhibitor |
| SEPRACOR | Lansoprazole | Lansoprazole | Acid | Proton pump |
| | (SD) | (SD) | Disorders | inhibitor |
| MERCK | Mepral | Omeprazole | Acid | Proton pump |
| KGAA | | | Disorders | inhibitor |
| SCHWARZ | Rifun | Pantoprazol | Acid | Proton pump |
| | | | Disorders | inhibitor |
| NYCOMED | Zurcal/ | Pantoprazol | Acid | Proton pump |
| AMERSHA | Pantaloc | | Disorders | inhibitor |
| ALTANA | Protonix/ | Pantoprazol | Acid | Proton pump |
| | Pantoloc | | Disorders | inhibitor |
| SEPRACOR | Pantoprazole | Pantoprazol | Acid | Proton pump |
| | (−) | (−) | Disorders | inhibitor |
| BASF | TU 199 | TU 199 | Acid | Proton pump |
| | | | Disorders | inhibitor |
| AHP | Zolon | Lansoprazole | Acid | Proton pump |
| | | | Disorders | inhibitor |
| TAKEDA | Takepron | Lansoprazole | Acid | Proton pump |
| | | | Disorders | inhibitor |
| TAKEDA | Zolon | Lansoprazole | Acid | Proton pump |
| | | | Disorders | inhibitor |
| TAKEDA | Pravacid | Lansoprazole | Acid | Proton pump |
| | | | Disorders | inhibitor |
| ASTRA | PriLosec | Omeprazole | Acid | Proton pump |
| | | | Disorders | inhibitor |
| ASTRA | Losec/Antra | Omeprazole | Acid | Proton pump |
| | | | Disorders | inhibitor |
| MERCK | PriLosec | Omeprazole | Acid | Proton pump |
| | | | Disorders | inhibitor |
| SCHERING | Omepral | Omeprazole | Acid | Proton pump |
| PLOUGH | | | Disorders | inhibitor |
| FUJISAWA | Omepral | Omeprazole | Acid | Proton pump |
| | | | Disorders | inhibitor |
| AHP | Protonix | Pantoprazole | Acid | Proton pump |
| | | | Disorders | inhibitor |
| DAIICHI | DZ-2352a | Pantoprazole | Acid | Proton pump |
| | | | Disorders | inhibitor |
| ASTRA | Perprazole | Perprazole | Acid | Proton pump |
| | | | Disorders | inhibitor |
| JOHNSON & | Actiphex | Rabeprazole | Acid | Proton pump |
| JOHNSON | | | Disorders | inhibitor |
| JOHNSON & | Pariet | Rabeprazole | Acid | Proton pump |
| JOHNSON | | | Disorders | inhibitor |
| EISAI | Pariet/ | Rabeprazole | Acid | Proton pump |
| | Aciphex | | Disorders | inhibitor |
| ASTRA | Losec | Losec | Acid | Proton pump |
| | follow up | follow up | Disorders | inhibitor- |
| | | | | reversible |
| MERCK | Losec | Losec | Acid | Proton pump |
| | follow up | follow up | Disorders | inhibitor- |
| | | | | reversible |
| MERCK | Perprazole | Perprazole | Peptic | Proton pump |
| | | | Ulcer/GERD | inhibitor |
| ASTRA | Mosapride | Mosapride | Prokinetic, | |
| | | | dyspepsia | |

TABLE 2

| Name | Active Components |
|---|---|
| Advanced Formula | Calcium Carbonate, Magnesium hydroxide |
| Di-Gel Tablets, USP | 128 mg, Simethicone 20 mg, 10 mMq, |
| | Sodium < 5 mg |
| Almag | Aluminum hydroxide 225 mg, Magnesium |
| Oral Suspension USP | hydroxide 200 mg, Sodium <1.25 mg, |
| | Sugar free |
| Almag Plus | Aluminum hydroxide 225 mg, Magnesium |
| Oral Suspension USP | hydroxide 200 mg, Simethicone 25 mg, |
| | Sodium <5 mg , Sugar free |
| Alenic Alka | Aluminum hydroxide 31.7 mg, Magnesium |
| Oral Suspension | hydroxide 137 mg, Sodium alginate, |
| | Sodium 13 mg. |
| Chewable Tablets | Aluminum hydroxide (dried gel) |
| | 80 mg, Magnesium trisilicate 20 mg, |
| | Alginic acid, Sodium 18.4 mg |
| Alenic Alka | Aluminum hydroxide 160 mg, |
| Extra Strength | Magneium carbonate 105 mg, |
| Tablets USP | Alginic acid, Sodium bicarbonate, |
| (Chewable) | Sodium 29.9 mg |
| Alka-Mints | Calcium carbonate 850 mg, 15.9 mEq, |
| Tablets USP | Sodium <0.5 mg |
| (Chewable) | |
| Alkets | Calcium carbonate 500 mg, Sodium ≦ mg |
| Tablets USP | |
| (Chewable) | |
| Alkets Extra Strength | Calcium carbonate 500 mg, Sodium ≦ mg |
| Tablets USP | |
| (Chewable) | |
| Almacone | Aluminum hydroxide (equiv. to dried gel) |
| Oral Suspension USP | 200 mg, Magnesium hydroxide 200 mg, |
| | Simethicone 20 mg, 10 mEq, Sodium 0.75 mg |
| Tablets USP | Aluminum hydroxide |
| (Chewable) | (dried gel) 200 mg, Magnesium hydroxide 200 |
| | mg, Simethicone 20 mg |
| Almacone II | Aluminum hydroxide 400 mg, Magnesium |
| Oral Suspension USP | hydroxide 200 mg, Simethicone 20 mg, |
| | 20 mEq, Sodium 1.5 mg |
| Almagel 200 | Aluminum hydroxide hydroxide (equiv. |
| Oral Suspension USP | to dried gel) 200 mg, Magnesium hydroxide |
| | 200 mg |
| AlternaGEL | Aluminum hydroxide (equiv. to dried gel) |
| Gel USP | 600 mg, Simethicone, 16 mEq, Sodium |
| | <2.5 mg, sugar free |
| Alu-Cap | Aluminum hydroxide (dried gel) 400 mg, |
| Capsules USP | 8.5 mEq |
| Aludrox | Aluminum hydroxide gel 307 mg, |
| Oral Suspension USP | Magnesium hydroxide 103 mg, Simethicone |
| | 5 mg, 12 mEq, Sodium 2 mg |
| Alugel | Aluminum hydroxide gel 320 mg |
| Gel USP | |
| Alumina and Magnesia | Aluminum hydroxide (equiv. to dried gel) |
| Oral Suspension USP | 225-240 mg, Magnesium hydroxide |
| | 200-210 mg, 13.3 mEq, Sugar free |
| Alumina, Magnesia | Aluminum hydroxide (equiv. to dried gel) |
| and Simethicone | 213-225, Magnesium hydroxide 200 mg, |
| Oral Suspension USP | Simethicone 20-25 mg, 12.7 mEq, Sugar free |
| Aluminum Hydroxide | Aluminum hydroxide gel 320-675 mg |
| Gel USP | |
| Aluminum Hydroxide | Aluminum hydroxide (dried gel) |
| Gel | 500-600 mg |
| Dried Tablets USP | |
| Alu-Tab | Aluminum hydroxide (dried gel) 500-600 mg, |
| Tablets USP | 10.6 mEq, Film-coated, Tartrazine free |
| Amitone | Calcium carbonate 350 mg, 7 mEq, |
| Tablets USP | Sodium <2 mg |
| Amphojel | Aluminum hydroxide gel 320 mg, 10 mEq, |
| Gel USP | Sodium <2.3 mg (peppermint) |
| Tablets USP | Aluminum hydroxide (dried gel) 300-600 mg), |
| | 8-16 mEq, Sodium 1.4-2.8 mg |
| Amphojel 500 | Aluminum hydroxide 500 mg, Magnesium |
| Oral Suspension USP | hydroxide 500 mg, 37 mEq, |
| | Sodium 3 mg, Tartrazine fee, Sugar Free |
| Amphojel Plus | Aluminum hydroxide 300 mg, Magnesium |
| Oral Suspension USP | hydroxide 300 mg, Simethicone 25 mg, |
| | Sodium 7 mg, Sugar free, |
| | Tartrazine free |
| Chewable Tablets | Magnesium hydroxide 300 mg, Aluminum |
| | hydroxide and magnesium carbonate |
| | co-dried gel 300 mg, Simethicone 25 mg, |
| | Sodium 10 mg, Sugar free, Tartrazine free |
| Antacid Gelcaps | Calcium carbonate 311 mg, Magnesium |
| Tablets USP | carbonate 232 mg |
| Antacid Liquid | Aluminum hydroxide (equiv to dried gel) |
| Oral Suspension USP | 200 mg, Magnesium hydroxide 200 mg, |
| | Simethicone 20 mg, Sodium <1.25 mg |
| Antacid Liquid | Aluminum hydroxide (equiv to dried gel) |
| Double Strength | 400 mg, Magnesium hydroxide 400 mg, |
| Oral Suspension USP | Simethicone 40 mg, Sodium <1.25 mg |
| Basiljel | Dried basic aluminum carbonate gel equiv. |
| Capsules | to 500 mg of aluminum hydroxide or 608 mg |
| | of dried aluminum hydroxide gel, |
| | 12 mEq, Sodium 2.76 mg |
| Oral Suspension | Basic aluminum carbonate gel |
| | equiv. to 400 mg of aluminum hydroxide, |
| | Simethicone 4 mg, 11.5 mEq, Sodium 3 mg |
| Tablets | Dried basic aluminum carbonate gel equiv. |
| | to 500 mg of aluminum hydroxide or 608 mg of |
| | dried aluminum hydroxide gel, 12.5 mEq, |
| | Sodium 2.76 mg |
| Calcium Carbonate | Calcium carbonate 1250 mg |
| Oral Suspension USP | Calcium carbonate 500-1250 mg |
| Tablets | Calcium carbonate 500-750 mg |
| Chewable Tablets | |
| Calglycine | Calcium carbonate 420 mg, |
| Tablets | Glycine 150 mg, Sugar free |
| Chooz | Calcium carboante 500 mg, 10 mEq, |
| Chewing Gum | Sodium <5 mg |
| Dicarbosil | Calcium carboante 500 mg, 10 mEq, |
| Chewable Tablets USP | Sodium <2 mg |
| Di-Gel | Aluminum hydroxide (dried gel) 200 mg, |
| Oral Suspension USP | Magnesium hydroxide 200 mg, Simethicone |
| | 20 mg, ≧9 mEq, Sodium ≦mg, Sugar free |
| Diovol | Aluminum hydroxide 165 mg, Magnesium |
| Oral Suspension | hydroxide 200 mg, 11.9 mEq, Alcohol |
| | 1%, Sodium <1 mg, Sugar free, Tartrazine free |
| Chewable Tablets | Magnesium hydroxide 100 mg, Aluminum |
| | hydroxide and magnesium carbonate |
| | co-dried gel 300 mg, 10 mEq, |
| | Sodium 1 mg, Sugar free, Tartrazine free |
| Diovol Caplets | Aluminum hydroxide (equiv. to dried gel) |
| Tablets | 200 mg, Magnesium hydroxide 200 mg, |
| | Sugar free, Tartrazine free |
| Diovol Ex | Aluminum hydroxide 494 mg, Magnesium |
| Oral Suspension | hydroxide 300 mg, 25 mEq, Alcohol 1%, |
| | Sodium <1 mg, Sugar free, Tartrazine free |
| Tablets | Aluminum hydroxide (equiv to dried gel) |
| | 600 mg, Magnesium hydroxide 300 mg, |
| | 24.6 mEq, Sodium 1 mg, Sugar free, |
| | Tartrazine free |
| Diovol Plus | Aluminum hydroxide 165 mg, Magnesium |
| Oral Suspension | hydroxide 200 mg, Simethicone |
| | 25 mg, 11.9 mEq, |
| | Alcohol <1%, Sodium <1 mg, |
| | Sugar free, Tartrazine free |
| Chewable Tablets | Magnesium hydroxide 100 mg, |
| | Aluminum hydroxide and magnesium |
| | carbonate co-dried gel 300 mg, |
| | Simethicone 25 mg, 10 mEq, Sodium 1 mg, |
| | Sugar free, Tartrazine free |
| Diovol Plus AF | Calcium carboante 200 mg, Magnesium |
| Oral Suspension | hydroxide 200 mg, Simethicone 25 mg, |
| | 9.8 mEq, Alcohol 1%, Sodium 1 mg, |
| | Sugar free, Tartrazine free |
| Chewable Tablets | Magnesium hydroxide 100 mg, Aluminum |
| | hydroxide and magnesium carbonate |
| | co-dried gel 300 mg, Simethicone |
| | 25 mg, 10 mEq, Sodium 1 mg, |
| | Sugar free, Tartrazine free |
| Equilet | Calcium carbonate 500 mg, Sodium 0.3 mg |
| Chewable Tablets USP | |
| Foamicon | Aluminum hydroxide 80 mg, Magnesium |
| Chewable Tablets USP | trisilicate 20 mg, Alginic acid, |
| | Sodium bicarbonate, Sodium 18.4 mg |
| Gasmas | Magnesium hydroxide 100 mg, Aluminum |
| Chewable Tablets | hydroxide and magnesium carbonate co- |
| | dried gel 3 00 mg, Simethicone 25 mg |
| Gaviscon | Aluminum hydroxide 31.7 mg, Magnesium |
| Oral Suspension USP | carbonate 119.3 mg, Sodium alginate, |
| | 2.5-4.3 mEq, Sodium 13 mg |
| Chewable Tablets USP | Aluminum hydroxide (dried gel) |
| | 80 mg, Magnesium trisilicate 20 mg, |
| | Alginic acid, Sodium bicarbonate, 0.5 mEq, |
| | Sodium 18.4 mg |
| Gaviscon-2 | Aluminum hydroxide (dried gel) 160 mg, |
| Chewable Tablets USP | Magnesium trisilicate 40 mg, |
| | Alginic acid, Sodium bicarbonate, |
| | 1 mEq, Sodium 36.8 mg |
| Gaviscon Acid | Calcium carbonate 660 mg, |
| Plus Gas Relief | Magnesium hydroxide 145 mg, |
| Oral Suspension/ | Simethicone 30 mg |
| Chewable Tablets USP | Calcium carbonate 585 mg, Magnesium |
| | hydroxide 120 mg, Simethicone 30 mg |
| Gaviscon Acid Relief | Calcium carbonate 660 mg, |
| Oral Suspension | Magnesium hydroxide 145 mg |
| Chewable Tablets USP | Calcium carbonate 585 mg, |
| | Magnesium hydroxide 120 mg |
| Gaviscon Extra | Calcium carbonate 1 gram, |
| Strength Acid Relief | Magnesium hydroxide 250 mg |
| Oral Suspension USP | |
| Gaviscon Extra | Aluminum hydroxide 254 mg, Magnesium |
| Strength Relief | carboante 238 mg, Sodium alginate, |
| Formula | Simethicone emulsion, 14.3 mEq, |
| Oral Suspension | Sodium 20.7 mg |
| Chewable Tablets | Aluminum hydroxide 160 mg, |
| | Magnesium carboante 105 mg, Alginic acid, |
| | Sodium bicarbonate, 5-7.5 mEq, |
| | Sodium 29.9 mg |
| Gaviscon Heartburn | Aluminum hydroxide (dried gel) 100 mg, |
| Relief | Magnesium carbonate 100 mg, |
| Oral Suspension USP | Sodium alginate 250 mg, Calcium |
| | carbonate, Sodium bicarbonate, Sodium |
| | 30 mg, Alcohol free, Sugar free, |
| | Tartrazine free |
| Chewable Tablets | Aluminum hydroxide |
| | (dried gel) 80 mg, Magnesium carbonate |
| | 40 mg, Alginic acid 200 mg, Sodium 22 mg, |
| | Tartrazine free |
| Gaviscon heartburn | Aluminum hydroxide (dried gel) 160 mg, |
| Relief Extra Strength | Alginic acid 400 mg, Tartrazine free |
| Chewable Tablets USP | |
| Gelusil | Aluminum hydroxide (equiv. to dried |
| Oral Suspension USP | gel) 200 mg, Magnesium hydroxide |
| | 200 mg, Sodium 0.84 mg, Sugar free, |
| | Tartrazine free |
| Chewable Tablets USP | Aluminum hydroxide |
| | (equiv. to dried gel) 200 mg, |
| | Magnesium hydroxide 200 mg, |
| | Simethicone 25 mg, 11 mEq, |
| | Sodium 5-1.1 mg, Tartrazine free. |
| Gelusil Extra | Aluminum hydroxide (equivalent to |
| Strength | dried gel) 650 mg, Magnesium |
| Oral Suspension USP | hydroxide 350 mg, Sodium 1.4 mg, |
| | Sugar free, Tartrazine free |
| Chewable Tablets USP | Aluminum hydroxide (equivalent to |
| | dried gel) 400 mg, Magnesium |
| | hydroxide 400 mg, Sodium 1.6 mg, |
| | Tartrazine free |
| Genaton | Aluminum hydroxide 31.7 mg, |
| Oral Suspension USP | Magnesium carbonate 137.3 mg, |
| | Sodium alginate, Sodium 13 mg |
| Chewable Tablets USP | Aluminum hydroxide 80 mg, Magnesium |
| | trisilicate 20 mg, Alginic |
| | acid, Sodium bicarbonate, Sodium 18.4 mg |
| Genaton Extra | Aluminum hydroxide 160 mg, |
| Strength | Magnesium carbonate 105 mg, |
| Chewable Tablets USP | Alginic acid, Sodium |
| | bicarbonate, Sodium 35 mg |
| Kudrox Double | Aluminum hydroxide 500 mg, |
| Strength | Mangesium hydroxide 450 mg, |
| Oral Suspension USP | Simethicone 40 mg, 25 mEq, |
| | Sodium <5 mg |
| Life Antacid | Aluminum hydroxide (dried gel) 228 mg, |
| Oral Suspension USP | Magnesium hydroxide 200 mg, Sugar free |
| Life Antacid Plus | Aluminum hydroxide (dried gel) 228 mg, |
| Oral Suspension USP | Magnesium hydroxide 200 mg, |
| | Simethicone 25 mg, Sugar free |
| Chewable Tablets USP | Aluminum hydroxide (dried gel) 200 mg, |
| | Magnesium hydroxide |
| | 200 mg, Simethicone 25 mg |
| Losopan | Magaldrate 540 mg, Sodium <5 mg |
| Oral Suspension USP | |
| Losopan Plus | Magaldrate 540 mg, Simethicone 40 mg, |
| Oral Suspension USP | Sodium <5 mg |
| Lowsium Plus | Magaldrate 540 mg, Simethicone 40 mg, |
| Oral Suspension USP | Sodium <5 mg |
| Maalox | Aluminum hydroxide (equiv. to dried gel) |
| Oral Suspension USP | 225 mg, Magnesium hydroxide 200 mg, |
| | 13.3 mEq, Sodium 0.92-1.5 mg, |
| | Sugar free, Tartrazine free |
| Chewable Tablets USP | Aluminum hydroxide (dried gel) 200-400 mg, |
| | Magnesium hydroxide 200-400 mg, |
| | 9.7 mEq, Sodium 0.7-0.93, |
| | Sugar free, Tartrazine free |
| Maalox Antacid | Calcium carboante 311 mg, |
| Caplets | Magnesium carbonate 232 mg |
| Tablets USP | |
| Maalox Heartburn | Magnesium carbonate 175 mg, |
| Relief Formula | Aluminum hydroxide-magnesium |
| Oral Suspension | carboante co-dried gel 140 mg, |
| | 8.5 mEq, Sodium <1.5 mg, |
| | Tartrazine |
| Maalox HRF | Magnesium alginate 250 mg, Magnesium |
| Oral Suspension USP | carboante 175 mg, Aluminum hydroxide- |
| | magnesium carbonate codried |
| | gel 140 mg, Sodium <5 mg, |
| | Sugar free, Tartrazine free |
| Chewable Tablets USP | Magnesium alginate 250 mg, |
| | Magnesium carboante 160 mg, |
| | Aluminum hydroxide-magnesium carbonate |
| | codried gel 180 mg, |
| | Sodium <3 mg, Tartrazine free |
| Maalox Plus | Aluminum hydroxide (equiv. to dried gel) |
| Oral Suspension USP | 225 mg, Magnesium hydroxide 200 mg, |
| | Simethicone 25 mg, 13.35 mEq, |
| | Sodium 0.92-1.5 mg, |
| | Sugar free, Tartrazine free |
| Chewable Tablets USP | Aluminum hydroxide (equiv. to dried gel) |
| | 200 mg, Magnesium hydroxide 200 mg, |
| | Simethicone 25 mg, 10.65 mEq, Sodium 1 mg |
| | (lemon), 0.94 (mint), Tartrazine free |
| Maalox Plus | Aluminum hydroxide (equiv. to dried gel) |
| Extra Strength | 500 mg, Magnesium hydroxide 450 mg, |
| Oral Suspension USP | Simethicone 40 mg, 26.1 mEq, |
| | Sodium <1-1.2 mg, |
| | Sugar free, Tartrazine free |
| Chewable Tablets USP | Aluminum hydroxide (dried gel) |
| | 350 mg, Magnesium hydroxide |
| | 350 mg, Simethicone 30 mg, |
| | 16.7 mEq, Sodium 1.4 mg, |
| | Sugar 0.72 gram |
| Maalox TC | Aluminum hydroxide (equiv. to dried gel) |
| Oral Suspension USP | 600 mg, Magnesium hydroxide 300 mg, |
| | 27.2 mEq, Sodium <1 mg, |
| | Sugar free, Tartrazine free |
| Chewable Tablets USP | Aluminum hydroxide (dried gel) |
| | 600 mg, Magnesium hydroxide |
| | 300 mg, 28 mEq, Sodium <0.98 mg, |
| | Tartrazine free |
| Magaldrate | Magaldrate 540 mg, Sodium free, |
| Oral Suspension USP | Sugare free, Dye free |
| Magaldrate and | Magaldrate 540 mg, Simethicone 20 mg |
| Simethicone | |
| Oral Suspension | |
| Magnalox | Aluminum hydroxide (equiv. to dried gel) |
| Oral Suspension USP | 225 mg, Magnesium hydroxide 450 mg, |
| | Simethicone, Sugar free |
| Magnalox Plus | Aluminum hydroxide (equiv. to dried gel) |
| Oral Suspension USP | 500 mg, Magnesium hydroxide 450 mg, |
| | Simethicone 40 mg |
| Magnesium Hydroxide | Magnesium hydroxide 285 mg, |
| Magnesia | Sugar Free |
| Chewable Tablets USP | |
| Milk of Magnesia USP | Magnesium hydroxide 400-440 mg, |
| | 14 mEq, Sugar free |
| Mag-Ox 400 | Magnesium oxide 400 mg, 20 mEq |
| Tablets USP | |
| Mallamint | Calcium carbonate 420 mg, |
| Chewable Tablets USP | Sodium <0.1 mg, Sugar free |
| Maox 420 | Magnesium oxide 420 mg, 21 mEq, |
| Tablets USP | Tartrazine |
| Marblen | Calcium carbonate 520 mg, Magnesium |
| Oral Suspension | carbonate 400 mg, 18 mEq, Sugar free |
| Tablets USP | Calcium carbonate 520 mg, Magnesium |
| | carbonate 400 mg, 18 mEq, Sugar free |
| Mi-Acid | Aluminum hydroxide (equiv. to dried gel) |
| Oral Suspension USP | 200 mg, Magnesium hydroxide 200 mg, |
| | Simethicone 20 mg, Sodium <5 mg. |
| Tablets USP | Calcium carbonate 311 mg, Magnesium |
| | hydroxide 232 mg |
| Mi-Acid | Aluminum hydroxide (equiv. to dried gel) |
| Double Strength | 400 mg, Magnesium hydroxide 400 mg, |
| Oral Suspension | Simethicone 40 mg, Sodium <5 mg. |
| Mintox | Aluminum hydroxide (equiv. to dried |
| Oral Suspension USP | gel) 225 mg, Magnesium hydroxide |
| | 200 mg, Sodium 1.38 mg |
| Chewable Tablets USP | Aluminum |
| | hydroxide 200 mg, |
| | Magnesium hydroxide 200 mg |
| Mintox Extra Strength | Aluminum hydroxide (equiv. to |
| Oral Suspension USP | dried gel) 500 mg, Magnesium |
| | hydroxide 450 mg, Simethicone |
| | 40 mg, Sodium <5 mg |
| Chewable Tablets USP | Aluminum hydroxide 200 mg, Magnesium |
| | hydroxide 200 mg, Simethicone |
| | 25 mg, |
| Mygel | Aluminum hydroxide (equiv. to dried |
| Oral Suspension USP | gel) 200 mg, Magnesium hydroxide |
| | 200 mg, Simethicone 20 mg, |
| | Sodium 1.38 mg |
| Mygell II | Aluminum hydroxide (equiv. to dried |
| Oral Suspension USP | gel) 400 mg, Magnesium hydroxide 400 mg, |
| | Simethicone 40 mg, Sodium 1.3 mg |
| Mylanta | Calcium carbonate 600 mg, 11.4 mEq |
| Lozenges | |
| Oral Suspension USP | Aluminum hydroxide (equiv. to dried gel) |
| | 200 mg, Magnesium hydroxide 200 mg, |
| | Simethicone 20 mg, 12.7 mEq, |
| | Sodium 0.68-3.2 mg, Sugar |
| | free, Tartrazine free |
| Chewable Tablets USP | Aluminum hydroxide (dried gel) 200 mg, |
| | Calcium carbonate 350 mg, |
| | Magnesium hydroxide 150-200 mg, |
| | Simethicone 20 mg, 12 mEq, |
| | Sodium 0.3-0.9, Tartrazine free |
| Mylanta Double | Aluminum hydroxide 400 mg, |
| Strength | Magnesium hydroxide 400 mg, |
| Oral Suspension USP | Simethicone 40 mg, 25.4 mEq, |
| | Sodium 1.14, Sugar free |
| Chewable Tablets USP | Aluminum hydroxide (equiv. to dried gel) |
| | 400 mg, Calcium carbonate 700 mg, |
| | Magnesium hydroxide 300-400 mg, |
| | Simethicone 30 mg, 24 mEq, Sodium |
| | 0.6-1.5, Tartrazine free |
| Mylanta Double | Aluminum hydroxide (equiv. to |
| Strength Plain | dried gel) 400 mg, Magnesium |
| Oral Suspension USP | hydroxide 400 mg, Sodium 10 mg, |
| | Sugar free, Tartrazine free |
| Mylanta Extra | Aluminum hydroxide (equiv. to |
| Strength | dried gel) 650 mg, Magnesium |
| Oral Suspension USP | hydroxide 350 mg, Simethicone |
| | 30 mg, Sodium 1.8 mg, |
| | Sugar free, Tartrazine free |
| Mylanta Gelcaps | Calcium carbonate 550 mg, Magnesium |
| Tablets | hydroxide 125 mg, 11.5 mEq, Benzyl |
| | alcohol, Sodium 2.5 mg |
| Nephrox | Aluminum hydroxide gel 320 mg, Mineral |
| Oral Suspension | Oil 10%, 9 mEq, Sugar |
| Neutralca-S | Aluminum hydroxide (equiv. to |
| Oral Suspension USP | dried gel) 200 mg, Magnesium |
| | hydroxide 200 mg, Sodium 0.6 mg, |
| | Sugar free |
| Chewable Tablets USP | Aluminum hydroxide (dried gel) |
| | 400 mg, Magnesium hydroxide 400 mg, |
| | Sodium 1.01 mg, Scored |
| Phillips’ | Magnesium hydroxide 311 mg, |
| Chewable Magnesia | Low sodium, Sucrose 195 mg |
| Tablets | |
| Milk of Magnesia USP | Magnesium hydroxide 400 mg, Alcohol |
| | free, Sodium <2.2 mg, |
| | Sugar free (plain and mint) |
| Phillips’ Chewable | Magnesium hydroxide 311 mg |
| Chewable Magnesia | |
| Tablets USP | |
| Phillips’ Concentrated | Magnesium hydroxide 800 mg |
| Double-strength | |
| Milk of Magnesia USP | |
| PMS Alumina, | Aluminum hydroxide 200 mg, |
| Magneia | Magnesium hydroxide 200 mg, |
| and Simethicone | Simethicone 25 mg, Sugar free |
| Oral Suspension USP | |
| Rafton | Aluminum hydroxide 100 mg, |
| Oral Suspension | Calcium carbonate, Sodium |
| | bicarbonate, Sodium alginate 250 mg, |
| | Alcohol free, Sucrose 1.2 gm, |
| | Tartrazine free |
| Chewable Tablets | Aluminum hydroxide (equiv. to dried gel) |
| | 80 mg, Alginic acid 200 mg, Sodium |
| | bicarbonate, Sodium 22 mg, |
| | Sucrose 1.2 gm, Tartrazine free |
| Riopan | Magaldrate 540 mg, 15 mEq, |
| Oral Suspension USP | Sodium <0.3 mg |
| Chewable Tablets USP | Magaldrate 480 mg, Alcohol free, |
| | Sodium <0.7 mg, Sugar free, |
| | Tartarzine Free |
| Riopan Extra Strength | Magaldrate 1080 mg, Alcohol free, |
| Oral Suspension USP | Sodium 0.3 mg, Sugar free, |
| | Tartarzine Free |
| Riopan Plus | Magaldrate 480-540 mg, Simethicone |
| Oral Suspension USP | 20-40 mg, 13.5-15 mEq, |
| | Sodium <0.3-0.7 mg, Sugar free, |
| | Tartrazine free |
| Chewable Tablets USP | Magaldrate 480 mg, Simethicone 20 mg, |
| | 13.5 mEq, Sodium 0.1 mg |
| Riopan Plus | Magaldrate 1080 mg, Simethicone |
| Double Strength | 40 mg, Sodium ≦0.3 mg |
| Oral Suspension USP | |
| Chewable Tablets USP | Magaldrate 1080 mg, Simethicone 20 mg, |
| | 30 mEq, Sodium ≦0.5 mg |
| Riopan Plus | Magaldrate 1080 mg, Simethicone 30 mg, |
| Extra Strength | 30 mEq, Sodium 0.3 mg, Sugar free, |
| Oral Suspension USP | Tartrazine free |
| Rolaids | Calcium carbonate 317-550 mg, |
| Chewable Tablets USP | Magnesium hydroxide 64-110 mg, |
| | 14.8 mEq, Sodium <1 mg, |
| | Tartrazine free |
| Rolaids Extra Strength | Calcium carbonate 750 mg, Magnesium |
| Chewable Tablets USP | hydroxide 64 mg, Sodium <1 mg, |
| | Tartrazine free |
| Rulox | Aluminum hydroxide (equiv. to dried gel) |
| Oral Suspension USP | 225 mg., Magnesium hydroxide 200 mg, |
| | 12 mEq, Sodium <1 mg |
| Rulox No. 1 | Aluminum hydroxide (dried gel) 200 mg., |
| Chewable Tablets USP | 200 mg Magnesium hydroxide |
| Rulox No. 2 | Aluminum hydroxide (dried gel) 400 mg., |
| Chewable Tablets USP | Magnesium hydroxide 400 mg |
| Rulox Plus | Aluminum hydroxide (equiv. to dried gel) |
| Oral Suspension USP | 500 mg., Magnesium hydroxide 450 mg, |
| | Simethicone 40 mg |
| Simaal Gel | Aluminum hydroxide (equiv. to dried gel) |
| Oral Suspension USP | 200 mg., Magnesium hydroxide 200 mg, |
| | Simethicone 20 mg, Sodium |
| | 1.4 mg, Sugar free |
| Simaal 2 Gel | Aluminum hydroxide (equiv. to dried gel) |
| Oral Suspension USP | 400 mg., Magnesium hydroxide 400 mg, |
| | Simethicone 40 mg, Sodium |
| | 1.84 mg, Sugar free |
| Tempo | Aluminum hydroxide 133 mg, Calcium |
| Chewable Tablets USP | carbonate 414 mg, Magnesium hydroxide |
| | 81 mg, Simethicone 20 mg, 14 mEq, |
| | Sodium 3 mg |
| Titralac | Calcium carbonate 420 mg, Glycine 183 mg, |
| Chewable Tablets USP | 7.5 mEq, Sodium 1.1 mg, Sugar free |
| Titralac Extra Strength | Calcium carbonate 750 mg, Glycine 321 mg, |
| Chewable Tablets USP | Sodium 1.1 mg, Sugar free |
| Titralac Plus | Calcium carbonate 500 mg, Simethicone |
| Oral Suspension USP | 20 mg Sodium 2.5 mg, Sugar free |
| Chewable Tablets USP | Calcium carbonate 420 mg, Glycine |
| | 173 mg, Simethicone 21 mg, |
| | Sodium 1.1 mg, Sugar free |
| Trial | Calcium carbonate 420 |
| Chewable Tablets USP | |
| Turns | Calcium carbonate 500 mg, 10 mEq, |
| Chewable Tablets USP | Sodium <2 mg |
| Turns Anti-gas/ | Calcium carbonate 500 mg, Simethicone |
| Antacid | 20 mg, 10 mEq, Sodium ≦2 mg |
| Chewable Tablets USP | |
| Turns E-X | Calcium carbonate 750 mg, 15 mEq, |
| Chewable Tablets USP | Sodium <2 mg |
| Turns Extra Strength | Calcium carbonate 750 mg, 15 mEq, |
| Chewable Tablets USP | Sodium <2 mg |
| Turns Ultra | Calcium carbonate 1 gram, 20 mEq, |
| Chewable Tablets USP | Sodium ≦4 mg |
| Univol | Aluminum hydroxide 165 mg, Magnesium |
| Oral Suspension | hydroxide 200 mg, Alcohol 1%, Sodium 1 mg, |
| | Sugar free, Tartrazine free |
| Uro-Mag | Magnesium oxide 140 mg, 7 mEq |
| Capsules USP | |

The anti-bacterial agent may be an antibiotic, such as a broad spectrum antibiotic, a narrow spectrum antibiotic, or a limited spectrum antibiotic. In some embodiments the anti-bacterial agent is a cell wall synthesis inhibitor, cell membrane inhibitor, protein synthesis inhibitor, nucleic acid synthesis or functional inhibitor, competitive inhibitor, amoxicillin; clarithromycin; amoxicillin/clarithromycin combination; metronidazole; tetracycline, or naphthyridine carboxylic acid antibacterial compounds, or combinations thereof.

The antacid in some embodiments includes, but is not limited to, aluminum hydroxide, aluminum carbonate, aluminum phosphate, calcium carbonate, magnesium oxide, magnesium hydroxide, magnesium carbonate, magnesium alginate, magnesium trisilicate, sodium bicarbonate, sodium alginate, magaldrate, simethicone, or combinations thereof.

The ulcer adherent complex in some embodiments includes, but is not limited to, an alpha-D glucopyranoside beta-D fructofuranosyl-octakis-(hydrogen sulfate) aluminum complex such as sucralfate.

The H₂ receptor blockers/antagonist in some embodiments includes, but is not limited to, nizatidine, famotidine, cimetidine, or ranitidine hydrochloride.

The proton pump inhibitor in some embodiments includes, but is not limited to, omeprazole, lansoprazole, or prevpac.

The anti-cholinergic in some embodiments includes, but is not limited to, atropine, belladonna, clidinium, hyoscyamine, pirenzepine, or propantheline.

Other treatments for ulcers using compounds or formulas described in patents and patent applications are as follows: ACE-inhibitors, oligosaccharides formulas as described in U.S. Pat. Nos. 5,883,079 and 5,514,660, somatosatin or somatostatin agonist as described in U.S. Pat. No. 5,968,903, naphthyridine carboxylic acid antibacterial compounds as described in U.S. Pat. Nos. 5,900,414 and 5,164,402, immunogenic compositions capable of inducing antibodies against Helicobacter pylori as described in U.S. Pat. No. 5,843,460, combination of an H₂ receptor blocker and an acid degradable antibacterial compound as described in U.S. Pat. Nos. 5,633,244, 5,629,305, and 5,599,794, flavone and flavone compounds as described in U.S. Pat. No. 6,025,387, imidazopyridazines as described in U.S. Pat. No. 6,043,242, dimethicone as described in U.S. Pat. No. 6,028,062, pyridine compounds as described in U.S. Pat. Nos. 5,616,581 and 5,504,082, monoglycerides of fatty acids and lauric acid as described in U.S. Pat. No. 5,660,842, N-substituted derivative of 2-(pyridylalkene sulfinyl)benzimidazoles as described in U.S. Pat. No. 4,873,337, extract of the plant Thymus as described in U.S. Pat. No. 5,472,695, diphenyl ether phosphate ester as described in U.S. Pat. No. 5,447,923, triclosan as described in U.S. Pat. No. 5,286,492, specific immunoglobulins (antibodies) derived from the mammary secretions of cows and other animals immunized with Helicobacter pylori as described in U.S. Pat. Nos. 5,260,057 and 5,258,178, salt of a basic histamine H₂-receptor antagonist and a complex of bismuth with a carboxylic acid selected from tartaric acid, citric acid and alkyl citric acids, or a solvate of such a salt as described in U.S. Pat. No. 5,229,418, sulfated glyceroglucolipid as described in U.S. Pat. No. 5,116,821, polypeptides isolated from Streptococcus pneumoniae as described in patents # EP 889132 A, EP 881297 A, EP 881296 A, EP 881295 A, and EP 881286, and polypeptides isolated from Staphylococcus aureus as described in EP patent applications EP893502, EP889132, EP889131, and EP/889129.

ACE-inhibitors as described in U.S. Pat. No. 5,977,159 include but are not limited to alacepril, alatriopril, altiopril calcium, ancovenin, benazepril, benazepril hydrochloride, benazeprilat, benzazepril, benzoylcaptopril, captopril, captopril-cysteine, captopril-glutathione, ceranapril, ceranopril, ceronapril, cilazapril, cilazaprilat, converstatin, delapril, delapril-diacid, enalapril, enalaprilat, enalkiren, enapril, epicaptopril., foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciurmin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril, zofenoprilat. Where applicable, a compound listed above may be used in racemic form or in the form of a pure or substantially pure enantiomer.

Other methods include administering a scavenging, reacting or inactivating compound to remove bicarbonate ions, ammonium ions or urea which are present in combination with the microorganisms which infect the gastric mucosa as described in U.S. Pat. No. 5,409,903.

Anti-sense oligonucleotides are described in patents U.S. Pat. No. 5,977,340, WO #9629399 A, WO # 9832467, WO # 9737044 A, WO # 9719098 A1, WO # 9629399 A, WO # 9629399, EP 815217 A1, and in JP 9095454 A. Gastrointestinal polynucleotides are described in patent WO #9844159 A1.

The compounds useful according to the invention are nucleic acids. The nucleic acids may be double-stranded or single-stranded. Generally, double-stranded molecules may be more stable in vivo, while single-stranded molecules may have increased activity. The terms “nucleic acid” and “oligonucleotide” refer to multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)) or a modified base. As used herein, the terms refer to oligoribonucleotides as well as oligodeoxyribonucleotides. The terms shall also include polynucleosides (i.e. a polynucleotide minus the phosphate) and any other organic base containing polymer. The terms “nucleic acid” and “oligonucleotide” also encompass nucleic acids or oligonucleotides with a covalently modified base and/or sugar. For example, they include nucleic acids having backbone sugars which are covalently attached to low molecular weight organic groups other than a hydroxyl group at the 3′ position and other than a phosphate group at the 5′ position. Thus modified nucleic acids may include a 2′-O-alkylated ribose group. In addition, modified nucleic acids may include sugars such as arabinose instead of ribose. Thus the nucleic acids may be heterogeneous in backbone composition thereby containing any possible combination of polymer units linked together such as peptide-nucleic acids (which have amino acid backbone with nucleic acid bases). In some embodiments the nucleic acids are homogeneous in backbone composition.

The substituted purines and pyrimidines of the nucleic acids include standard purines and pyrimidines such as cytosine as well as base analogs such as C-5 propyne substituted bases (Wagner et al., Nature Biotechnology 14:840-844, 1996). Purines and pyrimidines include but are not limited to adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties.

The nucleic acid is a linked polymer of bases or nucleotides. As used herein with respect to linked units of a nucleic acid, “linked” or “linkage” means two entities are bound to one another by any physicochemical means. Any linkage known to those of ordinary skill in the art, covalent or non-covalent, is embraced. Such linkages are well known to those of ordinary skill in the art. Natural linkages, which are those ordinarily found in nature connecting the individual units of a nucleic acid, are most common. The individual units of a nucleic acid may be linked, however, by synthetic or modified linkages.

Whenever a nucleic acid is represented by a sequence of letters it will be understood that the nucleotides are in 5′→3′ order from left to right and that “A” denotes adenosine, “C” denotes cytosine, “G” denotes guanosine, “T” denotes thymidine, and “U” denotes uracil unless otherwise noted.

Nucleic acid molecules useful according to the invention can be obtained from natural nucleic acid sources (e.g. genomic nuclear or mitochondrial DNA or cDNA), or are synthetic (e.g. produced by oligonucleotide synthesis). Nucleic acids isolated from existing nucleic acid sources are referred to herein as native, natural, or isolated nucleic acids. The nucleic acids useful according to the invention may be isolated from any source, including eukaryotic sources, prokaryotic sources, nuclear DNA, mitochondrial DNA, etc. Thus, the term nucleic acid encompasses both synthetic and isolated nucleic acids.

The term “isolated” as used herein refers to a nucleic acid which is substantially free of or which is separated from components which it is normally associated with in nature e.g., nucleic acids, proteins, lipids, carbohydrates or in vivo systems to an extent practical and appropriate for its intended use. In particular, the nucleic acids are sufficiently pure and are sufficiently free from other biological constituents of host cells so as to be useful in, for example, producing pharmaceutical preparations. Because an isolated nucleic acid of the invention may be admixed with a pharmaceutically-acceptable carrier in a pharmaceutical preparation, the nucleic acid may comprise only a small percentage by weight of the preparation. The nucleic acid is nonetheless substantially pure in that it has been substantially separated from the substances with which it may be associated in living systems. The nucleic acids can be produced on a large scale in plasmids, (see Sambrook, T., et al., “Molecular Cloning: A Laboratory Manual”, Cold Spring Harbor laboratory Press, New York, 1989) and separated into smaller pieces or administered whole. After being administered to a subject the plasmid can be degraded into oligonucleotides. One skilled in the art can purify viral, bacterial, eukaryotic, etc. nucleic acids using standard techniques, such as those employing restriction enzymes, exonucleases or endonucleases.

For use in the instant invention, the nucleic acids can be synthesized de novo using any of a number of procedures well known in the art. For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et. al., Nucl. Acid. Res. 14:5399-5407, 1986, ; Garegg et al., Tet. Let. 27:4055-4058, 1986, Gaffney et al., Tet. Let. 29:2619-2622, 1988). These chemistries can be performed by a variety of automated oligonucleotide synthesizers available in the market.

The nucleic acids, however, do not include expression vectors containing genes which encode H. pylori antigens, in some embodiments. In other embodiments, the nucleic acids are not H. pylori antisense oligonucleotides. H. pylori antisense oligonucleotides are described in patents, such as U.S. Pat. No. 5,977,340, PCT Publication Nos. WO96/29399, WO98/32467, WO97/37044, WO97/19098, WO96/29399, WO96/29399, EP/815217, and JP9095454. Nucleic acid sequences and/or encoded polypeptides from H. pylori are described, for instance, in U.S. Pat. No. 5,801,013, and PCT Published Patent Applications WO97/37044 and WO97/19098. In other embodiments, these expression vectors and antisense molecules are included within the definition of nucleic acids.

In some embodiments, the nucleic acids useful according to the invention are immunostimulatory nucleic acids. An immunostimulatory nucleic acid is any nucleic acid, as described above, which is capable of modulating an immune response. A nucleic acid which modulates an immune response is one which produces any form of immune stimulation, including, but not limited to, induction of a cytokine, B cell activation, T cell activation, monocyte activation. Immunostimulatory nucleic acids include, but are not limited to, CpG nucleic acids, T-rich nucleic acids, poly G nucleic acids, and nucleic acids having phosphate modified backbones, such as phosphorothioate backbones.

A “CpG nucleic acid” or a “CpG immunostimulatory nucleic acid” as used herein is a nucleic acid containing at least one unmethylated CpG dinucleotide (cytosine-guanine dinucleotide sequence, i.e. “CpG DNA” or DNA containing a 5′ cytosine followed by 3′ guanosine and linked by a phosphate bond) and activates a component of the immune system. The entire CpG nucleic acid can be unmethylated or portions may be unmethylated but at least the C of the 5′ CG 3′ must be unmethylated.

In one embodiment the invention provides a CpG nucleic acid represented by at least the formula:

5′N₁X₁CGX₂N₂3′

wherein X₁ and X₂ are nucleotides and N is any nucleotide and N₁ and N₂ are nucleic acid sequences composed of from about 0-25 N's each. In some embodiments X₁ is adenine, guanine, or thymine and/or X₂ is cytosine, adenine, or thymine. In other embodiments X₁ is cytosine and/or X₂ is guanine.

In other embodiments the CpG nucleic acid is represented by at least the formula:

5′N₁X₁X₂CGX₃X₄N₂3′

wherein X₁, X₂, X₃, and X₄ are nucleotides. In some embodiments, X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, CpT, ApT, TpG, ApG, CpG, TpC, ApC, CpC, TpA, ApA, and CpA; N is any nucleotide and N₁ and N₂ are nucleic acid sequences composed of from about 0-25 N's each. In some embodiments, X₁X₂ are GpA or GpT and X₃X₄ are TpT. In other embodiments X₁ or X₂ or both are purines and X₃ or X₄ or both are pyrimidines or X₁X₂ are GpA and X₃ or X₄ or both are pyrimidines.

In another embodiment the CpG nucleic acid has the sequence 5′TCN₁TX₁X₂CGX₃X₄3′.

Examples of CpG nucleic acids according to the invention include but are not limited to those listed in Table 3.

A “T rich nucleic acid” or “T rich immunostimulatory nucleic acid” is a nucleic acid which includes at least one poly T sequence and/or which has a nucleotide composition of greater than 25% T nucleotide residues and which activates a component of the immune system. A nucleic acid having a poly-T sequence includes at least four Ts in a row, such as 5′TTTT3′. Preferably the T rich nucleic acid includes more than one poly T sequence. In preferred embodiments the T rich nucleic acid may have 2, 3, 4, etc poly T sequences, such as SEQ ID NO:146. One of the most highly immunostimulatory T rich oligonucleotides discovered according to the invention is a nucleic acid composed entirely of T nucleotide residues, e.g., SEQ ID NO: 148. Other T rich nucleic acids have a nucleotide composition of greater than 25% T nucleotide residues, but do not necessarily include a poly T sequence. In these T rich nucleic acids the T nucleotide resides may be separated from one another by other types of nucleotide residues, i.e., G, C, and A. In some embodiments the T rich nucleic acids have a nucleotide composition of greater than 30%, 40%, 50%, 60%, 70%, 80%, 90%, and 99%, T nucleotide residues and every integer % in between. Preferably the T rich nucleic acids have at least one poly T sequence and a nucleotide composition of greater than 25% T nucleotide residues.

In one embodiment the T rich nucleic acid is represented by at least the formula:

5′X₁X₂TTTTX₃X₄3′

wherein X₁, X₂,X₃, and X₄ are nucleotides. In one embodiment X₁X₂ is TT and/or X₃X₄ is TT. In another embodiment X₁X₂ are any one of the following nucleotides TA, TG, TC, AT, AA, AG, AC, CT, CC, CA, CG, GT, GG, GA, and GC; and X₃X₄ are any one of the following nucleotides TA, TG, TC, AT, AA, AG, AC, CT, CC, CA, CG, GT, GG, GA, and GC.

In some embodiments it is preferred that the T-rich nucleic acid does not contain poly C (CCCC), poly A (AAAA), poly G (GGGG), CpG motifs, or multiple GGs. In other embodiments the T-rich nucleic acid includes these motifs. Thus in some embodiments of the invention the T rich nucleic acids include CpG dinucleotides and in other embodiments the T rich nucleic acids are free of CpG dinucleotides. The CpG dinucleotides may be methylated or unmethylated.

Examples of T rich nucleic acids that are free of CpG nucleic acids include but are not limited to those listed in Table 3. Examples of T rich nucleic acids that include CpG nucleic acids include but are not limited to those listed in Table 3.

Poly G containing nucleic acids are also immunostimulatory. A variety of references, including Pisetsky and Reich, 1993 Mol. Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J Biochem. 116, 991-994 also describe the immunostimulatory properties of poly G nucleic acids.

Poly G nucleic acids preferably are nucleic acids having the following formulas:

5′ X₁X₂GGGX₃X₄3′

wherein X₁, X₂, X₃, and X₄ are nucleotides. In preferred embodiments at least one of X₃ and X₄ are a G. In other embodiments both of X₃ and X₄ are a G. In yet other embodiments the preferred formula is 5′ GGGNGGG 3′, or 5′ GGGNGGGNGGG 3′ wherein N represents between 0 and 20 nucleotides. In other embodiments the Poly G nucleic acid is free of unmethylated CG dinucleotides. In other embodiments the poly G nucleic acid includes at least one unmethylated CG dinucleotide.

Nucleic acids having modified backbones, such as phosphorothioate backbones, also fall within the class of immunostimulatory nucleic acids. U.S. Pat. Nos. 5,723,335 and 5,663,153 issued to Hutcherson, et al. and related PCT publication WO95/26204 describe immune stimulation using phosphorothioate oligonucleotide analogues. These patents describe the ability of the phosphorothioate backbone to stimulate an immune response in a non-sequence specific manner.

The immunostimulatory nucleic acid may be any size of at least 6 nucleotides but in some embodiments are in the range of between 6 and 100 or in some embodiments between 8 and 35 nucleotides in size. Immunostimulatory nucleic acids can be produced on a large scale in plasmids. These may be administered in plasmid form or alternatively they can be degraded into oligonucleotides before administration.

“Palindromic sequence” shall mean an inverted repeat (i.e. a sequence such as ABCDEE′D′C′B′A′ in which A and A′ are bases capable of forming the usual Watson-Crick base pairs and which includes at least 6 nucleotides in the palindrome. In vivo, such sequences may form double-stranded structures. In one embodiment the nucleic acid contains a palindromic sequence. In some embodiments when the nucleic acid is a CpG nucleic acid, a palindromic sequence used in this context refers to a palindrome in which the CpG is part of the palindrome, and optionally is the center of the palindrome. In another embodiment the nucleic acid is free of a palindrome. A nucleic acid that is free of a palindrome does not have any regions of 6 nucleotides or greater in length which are palindromic. A nucleic acid that is free of a palindrome can include a region of less than 6 nucleotides which are palindromic.

A “stabilized nucleic acid molecule” shall mean a nucleic acid molecule that is relatively resistant to in vivo degradation (e.g. via an exo- or endo-nuclease). Stabilization can be a function of length or secondary structure. Nucleic acids that are tens to hundreds of kbs long are relatively resistant to in vivo degradation. For shorter nucleic acids, secondary structure can stabilize and increase their effect. For example, if the 3′ end of an oligonucleotide has self-complementarity to an upstream region, so that it can fold back and form a sort of stem loop structure, then the oligonucleotide becomes stabilized and therefore exhibits more activity.

Some stabilized oligonucleotides of the instant invention have a modified backbone. It has been demonstrated that modification of the oligonucleotide backbone provides enhanced activity of the nucleic acids when administered in vivo. Nucleic acids, including at least two phosphorothioate linkages at the 5′ end of the oligonucleotide and multiple phosphorothioate linkages at the 3′ end, preferably 5, may provide maximal activity and protect the oligonucleotide from degradation by intracellular exo- and endo-nucleases. Other modified oligonucleotides include phosphodiester modified oligonucleotide, combinations of phosphodiester and phosphorothioate oligonucleotide, methylphosphonate, methylphosphorothioate, phosphorodithioate, and combinations thereof. Each of these combinations and their particular effects on immune cells is discussed in more detail in PCT Published Patent Applications claiming priority to U.S. Ser. Nos. 08/738,652 and 08/960,774, filed on Oct. 30, 1996 and Oct. 30, 1997 respectively, the entire contents of which is hereby incorporated by reference. It is believed that these modified oligonucleotides may show more stimulatory activity due to enhanced nuclease resistance, increased cellular uptake, increased protein binding, and/or altered intracellular localization. Both phosphorothioate and phosphodiester nucleic acids are active in immune cells.

Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

For use in vivo, nucleic acids are preferably relatively resistant to degradation (e.g., via endo-and exo-nucleases). Secondary structures, such as stem loops, can stabilize nucleic acids against degradation. Alternatively, nucleic acid stabilization can be accomplished via phosphate backbone modifications. One type of stabilized nucleic acid has at least a partial phosphorothioate modified backbone. Phosphorothioates may be synthesized using automated techniques employing either phosphoramidate or H-phosphonate chemistries. Aryl-and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Other sources of nucleic acids useful according to the invention include standard viral and bacterial vectors, many of which are commercially available. In its broadest sense, a “vector” is any nucleic acid material which is ordinarily used to deliver and facilitate the transfer of nucleic acids to cells. The vector as used herein may be an empty vector or a vector carrying a gene which can be expressed. In the case when the vector is carrying a gene the vector generally transports the gene to the target cells with reduced degradation relative to the extent of degradation that would result in the absence of the vector. In this case the vector optionally includes gene expression sequences to enhance expression of the gene in target cells such as immune cells, but it is not required that the gene be expressed in the cell.

In general, vectors include, but are not limited to, plasmids, phagemids, viruses, other vehicles derived from viral or bacterial sources. Viral vectors are one type of vector and include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Some viral vectors are based on non-cytopathic eukaryotic viruses in which non-essential genes have been replaced with a nucleic acid to be delivered. Non-cytopathic viruses include retroviruses, the life cycle of which involves reverse transcription of genomic viral RNA into DNA.

Standard protocols for producing empty vectors or vectors carrying genes (including the steps of incorporation of exogenous genetic material into a plasmid, transfection of a packaging cell line with plasmid, production of recombinant retroviruses by the packaging cell line, collection of viral particles from tissue culture media, and/or infection of the target cells with viral particles) are provided in Kriegler, M., “Gene Transfer and Expression, A Laboratory Manual,” W.H. Freeman C.O., New York (1990) and Murry, E. J. Ed. “Methods in Molecular Biology,” vol. 7, Humana Press, Inc., Cliffton, N.J. (1991).

Other vectors include plasmid vectors. Plasmid vectors have been extensively described in the art and are well-known to those of skill in the art. See e.g., Sambrook et al., “Molecular Cloning: A Laboratory Manual,” Second Edition, Cold Spring Harbor Laboratory Press, 1989. In the last few years, plasmid vectors have been found to be particularly advantageous for delivering genes to cells in vivo because of their inability to replicate within and integrate into a host genome. Some plasmids, however, having a promoter compatible with the host cell, can express a peptide from a gene operatively encoded within the plasmid. Some commonly used plasmids include pBR322, pUC18, pUC19, pcDNA3.1, SV40, and pBlueScript. Other plasmids are well-known to those of ordinary skill in the art. Additionally, plasmids may be custom designed using restriction enzymes and ligation reactions to remove and add specific fragments of DNA.

It has recently been discovered that plasmids (empty or gene carrying) can be delivered to the immune system using bacteria. Modified forms of bacteria such as Salmonella can be transfected with the plasmid and used as delivery vehicles. The bacterial delivery vehicles can be administered to a host subject orally or by other administration means. The bacteria deliver the plasmid to immune cells, e.g. dendritic cells, probably by passing through the gut barrier. High levels of immune protection have been established using this methodology. Such methods of delivery are useful for the aspects of the invention utilizing systemic delivery of nucleic acid.

Some of the nucleic acids useful according to the invention and described herein are presented below in Table 3.

TABLE 3

| | |
|---|---|
| GCTAGACGTTAGCGT; | (SEQ ID NO: 1) |
| GCTAGATGTTAGCGT; | (SEQ ID NO: 2) |
| GCTAGACGTTAGCGT; | (SEQ ID NO: 3) |
| GCTAGACGTTAGCGT; | (SEQ ID NO: 4) |
| GCATGACGTTGAGCT; | (SEQ ID NO: 5) |
| ATGGAAGGTCCAGCGTTCTC; | (SEQ ID NO: 6) |
| ATCGACTCTCGAGCGTTCTC; | (SEQ ID NO: 7) |
| ATCGACTCTCGAGCGTTCTC; | (SEQ ID NO: 8) |
| ATCGACTCTCGAGCGTTCTC; | (SEQ ID NO: 9) |
| ATGGAAGGTCCAACGTTCTC; | (SEQ ID NO: 10) |
| GAGAACGCTGGACCTTCCAT; | (SEQ ID NO: 11) |
| GAGAACGCTCGACCTTCCAT; | (SEQ ID NO: 12) |
| GAGAACGCTCGACCTTCGAT; | (SEQ ID NO: 13) |
| GAGAACGCTGGACCTTCCAT; | (SEQ ID NO: 14) |
| GAGAACGATGGACCTTCCAT; | (SEQ ID NO: 15) |
| GAGAACGCTCCAGCACTGAT; | (SEQ ID NO: 16) |
| TCCATGTCGGTCCTGATGCT; | (SEQ ID NO: 17) |
| TCCATGTCGGTCCTGATGCT; | (SEQ ID NO: 18) |
| TCCATGACGTTCCTGATGCT; | (SEQ ID NO: 19) |
| TCCATGTCGGTCCTGCTGAT; | (SEQ ID NO: 20) |
| TCAACGTT; | (SEQ ID NO: 21) |
| TCAGCGCT; | (SEQ ID NO: 22) |
| TCATCGAT; | (SEQ ID NO: 23) |
| TCTTCGAA; | (SEQ ID NO: 24) |
| CAACGTT; | (SEQ ID NO: 25) |
| CCAACGTT; | (SEQ ID NO: 26) |
| AACGTTCT; | (SEQ ID NO: 27) |
| TCAACGTC; | (SEQ ID NO: 28) |
| ATGGACTCTCCAGCGTTCTC; | (SEQ ID NO: 29) |
| ATGGAAGGTCCAACGTTCTC; | (SEQ ID NO: 30) |
| ATCGACTCTCGAGCGTTCTC; | (SEQ ID NO: 31) |
| ATGGAGGCTCCATCGTTCTC; | (SEQ ID NO: 32) |
| ATCGACTCTCGAGCGTTCTC; | (SEQ ID NO: 33) |
| ATCGACTCTCGAGCGTTCTC; | (SEQ ID NO: 34) |
| TCCATGTCGGTCCTGATGCT; | (SEQ ID NO: 35) |
| TCCATGCCGGTCCTGATGCT; | (SEQ ID NO: 36) |
| TCCATGGCGGTCCTGATGCT; | (SEQ ID NO: 37) |
| TCCATGACGGTCCTGATGCT; | (SEQ ID NO: 38) |
| TCCATGTCGATCCTGATGCT; | (SEQ ID NO: 39) |
| TCCATGTCGCTCCTGATGCT; | (SEQ ID NO: 40) |
| TCCATGTCGTCCCTGATGCT; | (SEQ ID NO: 41) |
| TCCATGACGTGCCTGATGCT; | (SEQ ID NO: 42) |
| TCCATAACGTTCCTGATGCT; | (SEQ ID NO: 43) |
| TCCATGACGTCCCTGATGCT; | (SEQ ID NO: 44) |
| TCCATCACGTGCCTGATGCT; | (SEQ ID NO: 45) |
| GGGGTCAACGTTGACGGGG; | (SEQ ID NO: 46) |
| GGGGTCAGTCGTGACGGGG; | (SEQ ID NO: 47) |
| GCTAGACGTTAGTGT; | (SEQ ID NO: 48) |
| TCCATGTCGTTCCTGATGCT; | (SEQ ID NO: 49) |
| ACCATGGACGATCTGTTTCCCCTC; | (SEQ ID NO: 50) |
| TCTCCCAGCGTGCGCCAT; | (SEQ ID NO: 51) |
| ACCATGGACGAACTGTTTCCCCTC; | (SEQ ID NO: 52) |
| ACCATGGACGAGCTGTTTCCCCTC; | (SEQ ID NO: 53) |
| ACCATGGACGACCTGTTTCCCCTC; | (SEQ ID NO: 54) |
| ACCATGGACGTACTGTTTCCCCTC; | (SEQ ID NO: 55) |
| ACCATGGACGGTCTGTTTCCCCTC; | (SEQ ID NO: 56) |
| ACCATGGACGTTCTGTTTCCCCTC; | (SEQ ID NO: 57) |
| CACGTTGAGGGGCAT; | (SEQ ID NO: 58) |
| TCAGCGTGCGCC; | (SEQ ID NO: 59) |
| ATGACGTTCCTGACGTT; | (SEQ ID NO: 60) |
| TCTCCCAGCGGGCGCAT; | (SEQ ID NO: 61) |
| TCCATGTCGTTCCTGTCGTT; | (SEQ ID NO: 62) |
| TCCATAGCGTTCCTAGCGTT; | (SEQ ID NO: 63) |
| TCGTCGCTGTCTCCCCTTCTT; | (SEQ ID NO: 64) |
| TCCTGACGTTCCTGACGTT; | (SEQ ID NO: 65) |
| TCCTGTCGTTCCTGTCGTT; | (SEQ ID NO: 66) |
| TCCATGTCGTTTTTGTCGTT; | (SEQ ID NO: 67) |
| TCCTGTCGTTCCTTGTCGTT; | (SEQ ID NO: 68) |
| TCCTTGTCGTTCCTGTCGTT; | (SEQ ID NO: 69) |
| TCCTGTCGTTTTTTGTCGTT; | (SEQ ID NO: 70) |
| TCGTCGCTGTCTGCCCTTCTT; | (SEQ ID NO: 71) |
| TCGTCGCTGTTGTCGTTTCTT; | (SEQ ID NO: 72) |
| TCCATGCGTGCGTGCGTTTT; | (SEQ ID NO: 73) |
| TCCATGCGTTGCGTTGCGTT; | (SEQ ID NO: 74) |
| TCCACGACGTTTTCGACGTT; | (SEQ ID NO: 75) |
| TCGTCGTTGTCGTTGTCGTT; | (SEQ ID NO: 76) |
| TCGTCGTTTTGTCGTTTTGTCGTT; | (SEQ ID NO: 77) |
| TCGTCGTTGTCGTTTTGTCGTT; | (SEQ ID NO: 78) |
| GCGTGCGTTGTCGTTGTCGTT; | (SEQ ID NO: 79) |
| TGTCGTTTGTCGTTTGTCGTT; | (SEQ ID NO: 80) |
| TGTCGTTGTCGTTGTCGTTGTCGTT; | (SEQ ID NO: 81) |
| TGTCGTTGTCGTTGTCGTT; | (SEQ ID NO: 82) |
| TCGTCGTCGTCGTT; | (SEQ ID NO: 83) |
| TGTCGTTGTCGTT; | (SEQ ID NO: 84) |
| TCCATAGCGTTCCTAGCGTT; | (SEQ ID NO: 85) |
| TCCATGACGTTCCTGACGTT; | (SEQ ID NO: 86) |
| GTCGYT; | (SEQ ID NO: 87) |
| TGTCGYT; | (SEQ ID NO: 88) |
| AGCTATGACGTTCCAAGG; | (SEQ ID NO: 89) |
| TCCATGACGTTCCTGACGTT; | (SEQ ID NO: 90) |
| ATCGACTCTCGAACGTTCTC; | (SEQ ID NO: 91) |
| TCCATGTCGGTCCTGACGCA; | (SEQ ID NO: 92) |
| TCTTCGAT; | (SEQ ID NO: 93) |
| ATAGGAGGTCCAACGTTCTC; | (SEQ ID NO: 94) |
| GCTAGAGGGGAGGGT; | (SEQ ID NO: 95) |
| GCTAGATGTTAGGGG; | (SEQ ID NO: 96) |
| GCTAGAGGGGAGGGT; | (SEQ ID NO: 97) |
| GCTAGAGGGGAGGGT; | (SEQ ID NO: 98) |
| GCATGAGGGGGAGCT; | (SEQ ID NO: 99) |
| ATGGAAGGTCCAGGGGGCTC; | (SEQ ID NO: 100) |
| ATGGACTCTGGAGGGGGCTC; | (SEQ ID NO: 101) |
| ATGGACTCTGGAGGGGGCTC; | (SEQ ID NO: 102) |
| ATGGACTCTGGAGGGGGCTC; | (SEQ ID NO: 103) |
| ATGGAAGGTCCAAGGGGCTC; | (SEQ ID NO: 104) |
| GAGAAGGGGGGACCTTCCAT; | (SEQ ID NO: 105) |
| GAGAAGGGGGGACCTTCCAT; | (SEQ ID NO: 106) |
| GAGAAGGGGGGACCTTGGAT; | (SEQ ID NO: 107) |
| GAGAAGGGGGGACCTTCCAT; | (SEQ ID NO: 108) |
| GAGAAGGGGGGACCTTCCAT; | (SEQ ID NO: 109) |
| GAGAAGGGGCCAGCACTGAT; | (SEQ ID NO: 110) |
| TCCATGTGGGGCCTGATGCT; | (SEQ ID NO: 111) |
| TCCATGTGGGGCCTGATGCT; | (SEQ ID NO: 112) |
| TCCATGAGGGGCCTGATGCT; | (SEQ ID NO: 113) |
| TCCATGTGGGGCCTGCTGAT; | (SEQ ID NO: 114) |
| ATGGACTCTCCGGGGTTCTC; | (SEQ ID NO: 115) |
| ATGGAAGGTCCGGGGTTCTC; | (SEQ ID NO: 116) |
| ATGGACTCTGGAGGGGTCTC; | (SEQ ID NO: 117) |
| ATGGAGGCTCCATGGGGCTC; | (SEQ ID NO: 118) |
| ATGGACTCTGGGGGGTTCTC; | (SEQ ID NO: 119) |
| ATGGACTCTGGGGGGTTCTC; | (SEQ ID NO: 120) |
| TCCATGTGGGTGGGGATGCT; | (SEQ ID NO: 121) |
| TCCATGCGGGTGGGGATGCT; | (SEQ ID NO: 122) |
| TCCATGGGGGTCCTGATGCT; | (SEQ ID NO: 123) |
| TCCATGGGGGTCCTGATGCT; | (SEQ ID NO: 124) |
| TCCATGTGGGGCCTGATGCT; | (SEQ ID NO: 125) |
| TCCATGTGGGGCCTGATGCT; | (SEQ ID NO: 126) |
| TCCATGGGGTCCCTGATGCT; | (SEQ ID NO: 127) |
| TCCATGGGGTGCCTGATGCT; | (SEQ ID NO: 128) |
| TCCATGGGGTTCCTGATGCT; | (SEQ ID NO: 129) |
| TCCATGGGGTCCCTGATGCT; | (SEQ ID NO: 130) |
| TCCATCGGGGGCCTGATGCT; | (SEQ ID NO: 131) |
| GCTAGAGGGAGTGT; | (SEQ ID NO: 132) |
| GGGGGGGGGGGGGGGGGGGG; | (SEQ ID NO: 133) |
| ACTGACAGACTGACAGACTGA; | (SEQ ID NO: 134) |
| AGTGACAGACAGACACACTGA; | (SEQ ID NO: 135) |
| ACTGACAGACTGATAGACCCA; | (SEQ ID NO: 136) |
| AGTGAGAGACTGCAAGACTGA; | (SEQ ID NO: 137) |
| AATGCCAGTCCGACAGGCTGA; | (SEQ ID NO: 138) |
| CCAGAACAGAAGCAATGGATG; | (SEQ ID NO: 139) |
| CCTGAACAGAAGCCATGGATG; | (SEQ ID NO: 140) |
| GCAGAACAGAAGACATGGATG; | (SEQ ID NO: 141) |
| CCACAACACAAGCAATGGATA; | (SEQ ID NO: 142) |
| AAGCTAGCCAGCTAGCTAGCA; | (SEQ ID NO: 143) |
| CAGCTAGCCACCTAGCTAGCA; | (SEQ ID NO: 144) |
| AAGCTAGGCAGCTAACTAGCA; | (SEQ ID NO: 145) |
| GAGCTAGCAAGCTAGCTAGGA; | (SEQ ID NO: 146) |
| TCGTCGTTTTGTCGTTTTGTCGTT; | (SEQ ID NO: 147) |
| TTTTTTTTTTTTTTTTTTTTTTTT; | (SEQ ID NO: 148) |

The nucleic acids are delivered in effective amounts. The term “effective amount” of a nucleic acid refers to the amount necessary or sufficient to realize a desired biologic effect. For example, an effective amount which alone or in combination with other therapeutics, and in single or multiple dosages is effective for treatment or prevention of ulcers. For instance, when the subject is infected with H. pylori, an effective amount is that amount which prevents an increase in numbers of H. pylori or which decreases or eliminates all together H. pylori infection. This can be assessed using one of the many known diagnostic assays for H. pylori infection (such as those described above). If the subject is not infected with H. pylori then an effective amount is that amount which prevents H. pylori infection when the subject is exposed to H. pylori. Additionally, an effective amount may be that amount which prevents an increase or causes a decrease in a symptom of gastric ulcer, such as pain. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the type of gastric ulcer being treated or prevented, the particular nucleic acid being administered (e.g. the number of unmethylated CpG motifs or their location in the nucleic acid), the use of an anti-ulcer agent, the size of the subject, or the severity of the disease or condition. One of ordinary skill in the art can empirically determine the effective amount of a particular nucleic acid without necessitating undue experimentation.

Subject doses of the compounds described herein typically range from about 0.1 μg to 10 mg per administration, which depending on the application could be given daily, weekly, or monthly and any other amount of time therebetween. More typically local doses range from about 10 μg to 5 mg per administration, and most typically from about 100 μg to 1 mg, with 2-4 administrations being spaced hours, days or weeks apart. More typically, immune stimulant doses range from 1 μg to 10 mg per administration, and most typically 10 μg to 1 mg, with daily or weekly administrations. Subject doses of the compounds described herein for parenteral delivery, wherein the compounds are delivered without another therapeutic agent are typically 5 to 10,000 times higher than the effective local dose or for immune stimulant applications, and more typically 10 to 1,000 times higher, and most typically 20 to 100 times higher. More typically parenteral doses for these purposes range from about 10 μg to 5 mg per administration, and most typically from about 100 μg to 1 mg, with 2-4 administrations being spaced hours, days or weeks apart. In some embodiments, however, parenteral doses for these purposes may be used in a range of 5 to 10,000 times higher than the typical doses described above.

For any compound described herein the therapeutically effective amount can be initially determined from animal models, e.g. the animal models described herein. A therapeutically effective dose can also be determined from human data for CpG nucleic acids which have been tested in humans (human clinical trials have been initiated and the results publicly disseminated) and for compounds which are known to exhibit similar pharmacological activities, such as other anti-ulcer agents. Higher doses may be required for parenteral administration, as described above. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the nucleic acid can be administered to a subject by any mode that delivers the nucleic acid to a subject. “Administering” the pharmaceutical composition of the present invention may be accomplished by any means known to the skilled artisan. Some routes of administration include but are not limited to oral, intranasal, intratracheal, inhalation, ocular, vaginal, rectal, parenteral (e.g. intramuscular, intradermal, intravenous or subcutaneous injection) and direct injection.

For oral administration, the compounds (i.e., nucleic acids and optionally anti-ulcer agents) can be delivered alone without any pharmaceutical carriers or formulated readily by combining the active compound(s) with pharmaceutically acceptable carriers well known in the art. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions.

Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray, from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions may also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533, 1990, which is incorporated herein by reference.

The nucleic acids and/or anti-ulcer agents may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The nucleic acids or other therapeutics useful in the invention may be delivered in mixtures with additional anti-ulcer agent(s). A mixture may consist of several anti-ulcer agents in addition to the nucleic acid.

A variety of administration routes are available. The particular mode selected will depend, of course, upon the particular nucleic acids or anti-ulcer agents selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed above.

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories. Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The nucleic acid may be directly administered to the subject or may be administered in conjunction with a pharmaceutically acceptable carrier or a delivery vehicle. The nucleic acid and optionally other therapeutic agents may be administered alone (e.g. in saline or buffer) or using any delivery vehicles known in the art. One type of delivery vehicle is referred to herein as a nucleic acid delivery complex. A “nucleic acid delivery complex” shall mean a nucleic acid molecule associated with (e.g. ionically or covalently bound to; or encapsulated within) a targeting means (e.g. a molecule that results in higher affinity binding to target cell (e.g. dendritic cell surfaces and/or increased cellular uptake by target cells). Examples of nucleic acid delivery complexes include nucleic acids associated with: a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to reduce significant uncoupling prior to internalization by the target cell. However, the complex may be cleavable under appropriate conditions within the cell so that the nucleic acid may be released in a functional form.

The nucleic acids may be delivered by non-invasive methods as described above. Non-invasive delivery of compounds is desirable for treatment of children, elderly, animals, and even adults and also to avoid the risk of needle-stick injury. Delivery vehicles for delivering compounds to mucosal surfaces have been described and include but are not limited to: Cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); Liposomes (Childers et al., 1999, Michalek et al., 1989, 1992, de Haan 1995a, 1995b); Live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); Live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); Microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); Polymers (e.g. carboxymethylcellulose, chitosan) (Hamajima et al., 1998, Jabbal-Gill et al., 1998); Polymer rings (Wyatt et al., 1998); Proteosomes (Vancott et al., 1998, Lowell et al., 1988, 1996, 1997); Sodium Fluoride (Hashi et al., 1998); Transgenic plants (Tacket et al., 1998, Mason et al., 1998, Haq et al., 1995); Virosomes (Gluck et al., 1992, Mengiardi et al., 1995, Cryz et al., 1998); Virus-like particles (Jiang et al., 1999, Leibl et al., 1998).

The present invention is further illustrated by the following Examples, which in no way should be construed as further limiting.

### EXAMPLES

Materials and Methods:

Oligodeoxynucleotides

Native phosphodiester and phosphorothioate-modified ODN are purchased from Operon Technologies (Alameda, Calif.) and Hybridon Specialty Products (Milford, Mass.). ODN are tested for endotoxin using the LAL-assay (LAL-assay BioWhittaker, Walkersville, Md.; lower detection limit 0.1 EU/ml). For in vitro assays, ODN are diluted in TE-buffer (10 mM Tris, pH 7.0, 1 mM EDTA), and stored at −20° C. For in vivo use, ODN are diluted in phosphate buffered saline (0.1 M PBS, pH 7.3) and stored at 4° C. All dilutions are carried out using pyrogen-free reagents.

Animals

Many animal models of gastric ulcer have been developed. U.S. Pat. No. 5,625,124 describes a transgenic non-human animal model of gastric ulcer. More recent U.S. Pat. No. 6,040,495 describes a hairless mouse sensitive to H. pylori infection which has been demonstrated to be a useful model of gastric ulcer. The model is useful for identifying compounds for the treatment of H. pylori infection as well as ulcers. Other models include gnotobiotic piglets and beagle dogs which have been artificially infected by H. pylori. These animals develop gastric ulcers that are similar to that seen in children, and thus is useful as a model for gastric ulcers in children. Non-human primates have also been identified as being susceptible to H. pylori infection, which results in gastric ulcer similar to infected adult humans. Thus, these primates can serve as a model of adult human gastric ulcer. An additional mouse model of gastric ulcer is described in U.S. Pat. No. 5,985,243. This patent describes euthymic mice which have been infected by fresh isoletes of H. pylori obtained directly from human patients and which produces a gastric pathology similar to that observed in humans. Any of these models can be used according to the invention.

A mouse model described in U.S. Pat. No. 5,985,243, which has been developed using non-toxic strain SPM314 and SPM326, is used to test the effectivity of the nucleic acids described herein. The animals are administered a nucleic acid sample composed of oligonucleotide 2006 by an oral route or a vehicle control. Colonization of mice by H. pylori is then assessed at various time points ranging from 1 day to 1 month after treatment. The ability of the nucleic acid to reduce H. pylori colonization is assessed.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

What is claimed is:

1. A method for preventing or treating a gastric ulcer, comprising:
administering to a subject in need thereof an effective amount for preventing or treating a gastric ulcer of a nucleic acid.

2. The method of claim 1, wherein the nucleic acid is an immunostimulatory CpG nucleic acid having an unmethylated CpG motif.

3. The method of claim 1, wherein the nucleic acid is an immunostimulatory T-rich nucleic acid.

4. The method of claim 1, wherein the nucleic acid is an immunostimulatory poly G nucleic acid.

5. The method of claim 1, wherein the nucleic acid is isolated.

6. The method of claim 1, further comprising administering an anti-ulcer agent.

7. The method of claim 6, wherein the anti-ulcer agent is an anti-bacterial agent.

8. The method of claim 1, wherein the nucleic acid is not an H. pylori anti-sense nucleic acid.

9. The method of claim 1, wherein the nucleic acid has a modified backbone.

10. The method of claim 9, wherein the modified backbone is a phosphate backbone modification.

11. The method of claim 9, wherein the modified backbone is a peptide modified oligonucleotide backbone.

12. The method of claim 7, wherein the nucleic acid is an immunostimulatory nucleic acid.

13. The method of claim 7, wherein the anti-bacterial agent is an antibiotic.

14. The method of claim 7, wherein the anti-bacterial agent is a narrow spectrum antibiotic.

15. The method of claim 7, wherein the anti-bacterial agent is a limited spectrum antibiotic.

16. The method of claim 7, wherein the anti-bacterial agent is selected from the group consisting of cell wall synthesis inhibitors, cell membrane inhibitors, protein synthesis inhibitors, nucleic acid synthesis or functional inhibitors, and competitive inhibitors.

17. The method of claim 7, wherein the anti-bacterial agent is selected from the group consisting of amoxicillin; clarithromycin; amoxicillin/clarithromycin combination; metronidazole; tetracycline, and naphthyridine carboxylic acid antibacterial compounds.

18. The method of claim 6, wherein the anti-ulcer agent is a compound selected from the group consisting of antacid, ulcer adherent complex, H₂ receptor blockers/antagonist, proton pump (H⁺, K⁺-ATPase) inhibitor, anti-cholinergic, ACE-inhibitor.

19. The method of claim 18, wherein the anti-ulcer agent is an antacid.

20. The method of claim 19, wherein the antacid is selected from the group consisting of aluminum hydroxide, aluminum carbonate, aluminum phosphate, calcium carbonate, magnesium oxide, magnesium hydroxide, magnesium carbonate, magnesium alginate, magnesium trisilicate, sodium bicarbonate, sodium alginate, magaldrate, simethicone, and combinations thereof.

21. The method of claim 18, wherein the anti-ulcer agent is an ulcer adherent complex.

22. The method of claim 21, wherein the ulcer adherent complex is an alpha-D glucopyranoside beta-D fructofuranosyl-octakis-(hydrogen sulfate) aluminum complex such as Sucralfate.

23. The method of claim 18, wherein the anti-ulcer agent is an H₂ receptor blockers/antagonist.

24. The method of claim 23, wherein the H₂ receptor blockers/antagonist is selected from the group consisting of nizatidine (Axid), famotidine (Pepcid: tablets, suspension, or injection; Pepcid AC), cimetidine (Tagamet: tablets, liquid, or injection), and ranitidine hydrochloride (Zantac:tablets, effervescent tablets, gel capsule, syrup, and injection).

25. The method of claim 18, wherein the anti-ulcer agent is a proton pump inhibitor.

26. The method of claim 25, wherein the proton pump inhibitor is selected from the group consisting of omeprazole, lansoprazole, and prevpac.

27. The method of claim 18, wherein the anti-ulcer agent is an anti-cholinergic.

28. The method of claim 27, wherein the anti-cholinergic is selected from the group consisting of atropine, belladonna, clidinium, hyoscyamine, pirenzepine, and propantheline.

29. The method of claim 18, wherein the anti-ulcer agent is an ACE-inhibitor.

30. The method of claim 29, wherein the ACE-inhibitor is selected from the group consisting of alacepril, alatriopril, altiopril calcium, ancovenin, benazepril, benazepril hydrochloride, benazeprilat, benzazepril, benzoylcaptopril, captopril, captopril-cysteine, captopril-glutathione, ceranapril, ceranopril, ceronapril, cilazapril, cilazaprilat, converstatin, delapril, delapril-diacid, enalapril, enalaprilat, enalkiren, enapril, epicaptopril., foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciurmin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril, zofenoprilat, racemic forms thereof, and pure or substantially pure enantiomers thereof.

31. The method of claim 6, wherein the anti-ulcer agent is a compound selected from the group consisting of an oligosaccharide, a somatosatin, a somatostatin agonist, a combination of an H2 receptor blocker and an acid degradable antibacterial compound, a flavone compound, an imidazopyridazine, a dimethicone, a pyridine compound, a monoglyceride of fatty acids and lauric acid, an N-substituted derivative of 2-(pyridylalkene sulfinyl)benzimidazole, a thymus plant extract, a diphenyl ether phosphate ester, a triclosan, anti-Helicobacter pylori immunoglobulin, a salt of a basic histamine H2 -receptor antagonist or a solvate thereof, a complex of bismuth with a carboxylic acid, a sulfated glyceroglucolipid, and a polypeptide isolated from Streptococcus pneumoniae and Staphylococcus aureus.

32. The method of claim 2, wherein the CpG nucleic acid comprises:
5′X₁ X₂CGX₃ X₄ 3′
wherein C is unmethylated, wherein X₁X₂ and X₃X₄ are nucleotides.

33. The method of claim 32, wherein the 5′ X₁ X₂CGX₃ X₄ 3′ sequence is a non-palindromic sequence.

34. The method of claim 32, wherein the CpG nucleic acid has 8 to 100 nucleotides.

35. The method of claim 32, wherein X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, CpT, ApT, TpG, ApG, CpG, TpC, ApC, CpC, TpA, ApA, and CpA.

36. The method of claim 32, wherein X₁X₂ are selected from the group consisting of GpA and GpT and X₃X₄ are TpT.

37. The method of claim 32, wherein X₁X₂ are both purines and X₃X₄ are both pyrimidines.

38. The method of claim 32, wherein X₂ is a T and X₃ is a pyrimidine.

39. The method of claim 32, wherein the CpG nucleic acid is 8 to 40 nucleotides in length.

40. The method of claim 3, wherein the T-rich nucleic acid is a poly T nucleic acid comprising
5′ TTTT 3′.

41. The method of claim 40, wherein the poly T nucleic acid comprises
5′ X₁ X₂TTTTX₃ X₄ 3′
wherein X₁, X₂, X₃ and X₄ are nucleotides.

42. The method of claim 40, wherein the T rich nucleic acid comprises a plurality of poly T nucleic acid motifs.

43. The method of claim 41, wherein X₁X₂ is TT.

44. The method of claim 41, wherein X₃X₄ is TT.

45. The method of claim 41, wherein X₁X₂is selected from the group consisting of TA, TG, TC, AT, AA, AG, AC, CT, CC, CA, CG, GT, GG, GA, and GC.

46. The method of claim 41, wherein X₃X₄ is selected from the group consisting of TA, TG, TC, AT, AA, AG, AC, CT, CC, CA, CG, GT, GG, GA, and GC.

47. The method of claim 41, wherein the T rich nucleic acid comprises a nucleotide composition of greater than 25% T.

48. The method of claim 3, wherein the T rich nucleic acid comprises a nucleotide composition of greater than 25% T.

49. The method of claim 48, wherein the T rich nucleic acid comprises a nucleotide composition of greater than 30% T.

50. The method of claim 48, wherein the T rich nucleic acid comprises a nucleotide composition of greater than 50% T.

51. The method of claim 48, wherein the T rich nucleic acid comprises a nucleotide composition of greater than 60% T.

52. The method of claim 48, wherein the T rich nucleic acid comprises a nucleotide composition of greater than 80% T.

53. The method of claim 3, wherein the T rich nucleic acid comprises at least 20 nucleotides.

54. The method of claim 3, wherein the T rich nucleic acid comprises at least 24 nucleotides.

55. The method of claim 4, wherein the poly G nucleic acid comprises:
5′ X₁X₂GGGX₃X₄ 3′
wherein X₁, X₂, X₃, and X₄ are nucleotides.

56. The method of claim 55, wherein at least one of X₃ and X₄ are a G.

57. The method of claim 55, wherein both of X₃ and X₄ are a G.

58. The method of claim 4, wherein the poly G nucleic acid comprises the following formula:
5′ GGGNGGG 3′
wherein N represents between 0 and 20 nucleotides.

59. The method of claim 4, wherein the poly G nucleic acid comprises the following formula:
5′ GGGNGGGNGGG 3′
wherein N represents between 0 and 20 nucleotides.

60. The method of claim 4, wherein the poly G nucleic acid is free of unmethylated CG dinucleotides

61. The method of claim 4, wherein the poly G nucleic acid includes at least one unmethylated CG dinucleotide.

62. The method of claim 1, wherein the nucleic acid is a synthetic nucleic acid.

63. The method of claim 6, wherein the nucleic acid is administered on a routine schedule.

64. The method of claim 63, wherein the anti-ulcer agent is administered on a routine schedule.

65. A composition, comprising:
a nucleic acid and an anti-ulcer agent, formulated in a pharmaceutically-acceptable carrier and in an effective amount for preventing or treating an ulcer.

66. The composition of claim 65, wherein the immunostimulatory nucleic acid has a modified backbone.

67. The composition of claim 66, wherein the modified backbone is a phosphate modified backbone.

68. The composition of claim 67, wherein the phosphate modified backbone is a phosphorothioate modified backbone.

69. The composition of claim 65, wherein the anti-ulcer agent is an antibiotic is selected from the group consisting of broad spectrum antibiotics, narrow spectrum antibiotics, and limited spectrum antibiotics.

70. The composition of claim 65, wherein the nucleic acid is an immunostimulatory CpG nucleic acid.

71. The composition of claim 65, wherein the nucleic acid is an immunostimulatory T-rich nucleic acid.

72. The composition of claim 65, wherein the nucleic acid is an immunostimulatory poly G nucleic acid.

73. The composition of claim 65, wherein the nucleic acid is isolated.

74. The composition of claim 65, wherein the anti-ulcer agent is not an anti-bacterial agent.

75. The composition of claim 65, wherein the anti-ulcer agent is a compound selected from the group consisting of antacid, ulcer adherent complex, H2 receptor blockers/antagonist, proton pump inhibitor, anti-cholinergic, ACE-inhibitor.

76. The composition of claim 65, wherein the anti-ulcer agent is an antacid.

77. The composition of claim 65, wherein the antacid is selected from the group consisting of aluminum hydroxide, aluminum carbonate, aluminum phosphate, calcium carbonate, magnesium oxide, magnesium hydroxide, magnesium carbonate, magnesium alginate, magnesium trisilicate, sodium bicarbonate, sodium alginate, magaldrate, simethicone, and combinations thereof.

78. The composition of claim 65, wherein the anti-ulcer agent is an ulcer adherent complex.

79. The composition of claim 65, wherein the ulcer adherent complex is an alpha-D glucopyranoside beta-D fructofuranosyl-octakis-(hydrogen sulfate) aluminum complex such as sucralfate.

80. The composition of claim 65, wherein the anti-ulcer agent is an H₂ receptor blockers/antagonist.

81. The composition of claim 65, wherein the H₂ receptor blockers/antagonist is selected from the group consisting of nizatidine (Axid), famotidine (Pepcid: tablets, suspension, or injection; Pepcid AC), cimetidine (Tagamet: tablets, liquid, or injection), and ranitidine hydrochloride (Zantac:tablets, effervescent tablets, gel capsule, syrup, and injection).

82. The composition of claim 65, wherein the anti-ulcer agent is a proton pump inhibitor.

83. The composition of claim 65, wherein the proton pump inhibitor is selected from the group consisting of omeprazole, lansoprazole, and prevpac.

84. The composition of claim 65, wherein the anti-ulcer agent is an Anti-cholinergic.

85. The composition of claim 65, wherein the anti-cholinergic is selected from the group consisting of atropine, belladonna, clidinium, hyoscyamine, pirenzepine, and propantheline.

86. The composition of claim 65, wherein the anti-ulcer agent is an ACE-inhibitor.

87. The composition of claim 65, wherein the ACE-inhibitor is selected from the group consisting of alacepril, alatriopril, altiopril calcium, ancovenin, benazepril, benazepril hydrochloride, benazeprilat, benzazepril, benzoylcaptopril, captopril, captopril-cysteine, captopril-glutathione, ceranapril, ceranopril, ceronapril, cilazapril, cilazaprilat, converstatin, delapril, delapril-diacid, enalapril, enalaprilat, enalkiren, enapril, epicaptopril., foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciurmin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril, zofenoprilat, racemic forms thereof, and pure or substantially pure enantiomers thereof.

88. The composition of claim 65, wherein the anti-ulcer agent is a compound selected from the group consisting of an oligosaccharide, a somatosatin, a somatostatin agonist, a combination of an H2 receptor blocker and an acid degradable antibacterial compound, a flavone compound, an imidazopyridazine, a dimethicone, a pyridine compound, a monoglyceride of fatty acids and lauric acid, an N-substituted derivative of 2-(pyridylalkene sulfinyl)benzimidazole, a thymus plant extract, a diphenyl ether phosphate ester, a triclosan, anti-Helicobacter pylori immunoglobulin, a salt of a basic histamine H₂ -receptor antagonist or a solvate thereof, a complex of bismuth with a carboxylic acid, a sulfated glyceroglucolipid, and a polypeptide isolated from Streptococcus pneumoniae and Staphylococcus aureus.

89. A kit comprising
at least one container housing nucleic acid, an anti-ulcer agent, and
instructions for administering the nucleic acid and the anti-ulcer agent to a subject having an ulcer or at risk of developing an ulcer.

90. The kit of claim 89, wherein the nucleic acid has a modified backbone.

91. The kit of claim 90, wherein the modified backbone is a phosphate modified backbone.

92. The kit of claim 91, wherein the phosphate modified backbone is a phosphorothioate modified backbone.

93. The kit of claim 89, wherein the anti-ulcer agent is an anti-bacterial agent.
